(19) United States
(12) Patent Application Publication
(10) Pub. No.: US 2017/0029278 A1
(43) Pub. Date: Feb. 2, 2017

(54) PROCESS FOR PREPARING SINGLE WALL CARBON NANOTUBES OF PRE-DEFINED CHIRALITY

(71) Applicants: Max-Planck-Gesellschaft, Zur Förderung der Wissenschaften e.V., München (DE); EMPA - Eidgenössische Materialprüfungs- und Forschungsanstalt, Dübendorf (CH)

(72) Inventors: Roman FASEL, Zürich (CH); Pascal RUFFIEUX, Plasselb (CH); Juan Ramon SANCHEZ VALENCIA, Malaga (ES); Martin JANSEN, Bonn (DE); Andreas MÜLLER, Quierschied (DE); Konstantin AMSHAROV, Erlangen (DE)

(73) Assignees: Max-Planck-Gesellschaft zur Forderung der Wissenschaften e.V., Munchen (DE); EMPA - Eldgenossische Materialprufungs - und Forschungsanstalt, Dubendorf (CH)

(21) Appl. No.: 15/303,139

(22) PCT Filed: Apr. 14, 2015
(86) PCT No.: PCT/EP2015/058058

§ 371 Date: Oct. 10, 2016

(30) Foreign Application Priority Data
Apr. 15, 2014 (EP) 14164733.9

(51) Int. Cl. C01B 31/02; C23C 16/44; C07C 15/20; C23C 16/26; B01J 23/42; C07C 13/62

(52) U.S. Cl. CPC C01B 31/0233; B01J 23/42; C07C 13/62; C07C 15/20; C23C 16/26; C23C 16/44; C01B 2202/02; B82Y 40/00

(57) ABSTRACT

The present invention relates to a process for preparing single wall carbon nanotubes (SWCNT) having a diameter d_{SWCNT}, which comprises
(i) providing a precursor element which comprises a segment S_{SWCNT} of the single wall carbon nanotube,
   the segment S_{SWCNT} being made of at least one ring formed by ortho-fused benzene rings, and having a first end E1 which is open and a second end E2 which is opposite to the first end E1,
(ii) growing the precursor element by vapour phase reaction with a carbon-source compound on the surface of a metal-containing catalyst, wherein the precursor element is in contact with the surface of the metal-containing catalyst via the open end E1 of the segment S_{SWCNT}, and the metal-containing catalyst is in the form of particles having an average diameter d_{cat} satisfying the following relation: d_{cat}>2×d_{SWCNT} or in the form of a continuous film.

Single-Walled Carbon Nanotubes (SWCNTs) represent an extended family of quasi one-dimensional nanostructures with tubular shape, composed of sp²-hybridised carbon atoms exclusively. Over the last decade SWCNTs have received tremendous attention from almost all areas of science because of their extraordinary thermal, mechanical, electronic and optical properties, and many potential high-technology applications. These materials are believed to be the best candidates that could revolutionize existing microelectronic by pushing it towards the nanometer scale. The diversity in properties which vary from semiconducting to conducting behaviour strongly depends on the orientation of the hexagonal lattice relative to the tube axis, as defined by the chiral indices (n, m) (chirality) and on the SWCNT diameter. Unfortunately, the wide application and exploring the potential of SWCNT for device performances are largely impeded by limited availability of uniform isomerically pure carbon nanotubes. SWCNTs are currently produced as highly heterogeneous mixture containing nanotubes of many different chiralities and diameters, regardless of the production technique used. Despite the considerable progress made in the past years in the development of sophisticated purification methods, a facile and effective separation of isomerically pure SWCNT suitable for large-scale technological processes remains still elusive.

One approach for preparing SWCNTs is by a vapour phase reaction of carbon-source molecules such as ethanol or ethylene on a catalyst surface (also referred to as chemical vapour deposition CVD). For initiating spontaneous reaction of the carbon-source molecules and, consequently, growth of SWCNTs on the catalyst surface, the catalyst particles need to have a diameter which corresponds more or less to the diameter of the SWCNTs to be prepared. Furthermore, these CVD processes are typically carried out at quite high temperature. Preparation of SWCNTs by vapour phase reaction needs the presence of nano-sized catalyst particles as described e.g. by Y. Homma et al., Nano Letters, Vol. 6, No. 12, 2006, pp. 2642-2645, and T. Maruyama et al., Mater. Express, Vol. 1, No. 4, 2011, pp. 267-272.

As discussed by R. Herges et al. in Chapter 10 (“Aromatic Belts as Sections of Nanotubes”) of Fragments of Fullerenes and Carbon Nanotubes, 2012, John Wiley & Sons, several approaches for a template-based CNT synthesis have been developed such as starting from nanotube caps or nanotube belts. For CNT growing strategies, different approaches are mentioned including inter alia metal-catalyzed vapour phase reaction (CVD). It is described that the catalyst particles used in such metal-catalyzed vapour phase reactions have a diameter which is about the diameter of the carbon nanotube to be prepared.

US 2012/0177561 A1 describes the preparation of SWCNTs by converting a polycyclic aromatic compound to an end cap of a SWCNT, said end cap acting as a SWCNT precursor element which is then grown to the final SWCNT in a Diels-Alder reaction, which is carried out in a solvent, preferably in the absence of a metal catalyst.

K. Amsharov and A. Müller, Eur. J. Org. Chem., 2012, 6155-6164 describe the preparation of polycyclic aromatic compounds that might be useful for preparing SWCNT precursor elements. However, neither the preparation of the precursor elements nor the growth of precursor elements to SWCNTs is actually described.

It is an object of the present invention to provide single wall carbon nanotubes (SWCNTs) of pre-defined chirality in high isomeric purity.

According to a first aspect of the present invention, the object is solved by a process for preparing single wall carbon nanotubes (SWCNTs) having a diameter d_{SWCNT}, which comprises
(i) providing a precursor element which comprises a segment S_{SWCNT} of the single wall carbon nanotube,
   the segment S_{SWCNT} being made of at least one ring formed by ortho-fused benzene rings, and having a first end E1 which is open and a second end E2 which is opposite to the first end E1,
   the precursor element optionally further comprising a cap which is attached to the second end E2 of the segment S_{SWCNT},
(ii) growing the precursor element by vapour phase reaction with a carbon-source compound on the surface of a metal-containing catalyst, wherein the precursor element is in contact with the surface of the metal-containing catalyst via the open end E1 of the segment S_{SWCNT}, and the metal-containing catalyst is in the form of particles having an average diameter d_{cat} satisfying the following relation: d_{cat}>2×d_{SWCNT} or in the form of a continuous film.

In the present invention, it has surprisingly been realized that selective growth of a precursor element to SWCNTs of pre-defined chirality by vapor phase reaction does not need to be carried out in the presence of catalyst particles having a diameter comparable to the SWCNT diameter but can be efficiently accomplished in the presence of larger catalyst particles (which do not need to have a narrow particle size distribution) or even continuous catalyst films, if said precursor element comprises a nanotube segment S_{SWCNT} being made of at least one ring formed by ortho-fused benzene rings, and the nanotube segment S_{SWCNT} is brought into contact with the metal catalyst via its open end E1 while the other end of the nanotube segment S_{SWCNT} can be open or closed by a SWCNT cap. As also discussed below, by using such a precursor element for vapor phase growth, it is not only possible to use CVD catalyst particles of larger size or even continuous catalyst films but also to improve yield at low growth temperature. Furthermore, using larger catalyst particles in step (ii) minimizes the risk or even excludes that the carbon-source compounds are spontaneously generating CNTs of undefined structure on the catalyst surface and makes sure that these carbon-source compounds are exclusively reacting with the SWCNT precursor element already provided in step (i).

As known to the skilled person, a nanotube is specified by the chiral vector C=na1+ma2 which represents the rolling direction of the graphene sheet and hence the CNT chirality. Accordingly, the structure of a carbon nanotube can be represented by a pair of integers (n,m). If m=0, the carbon nanotubes are so-called zigzag carbon nanotubes. If n=m, the carbon nanotubes are so-called armchair carbon nanotubes. Otherwise, the carbon nanotubes are classified as chiral.

With the process of the present invention, it is possible to obtain any of these single wall carbon nanotube structures. In other words, the single wall carbon nanotubes (SWCNTs) of the present invention can be armchair single wall carbon nanotubes ((n,n)-SWCNTs), zig-zag single wall carbon nanotubes ((n,0)-SWCNTs) or chiral single wall carbon nanotubes ((n,m)-SWCNTs with n≠m).

If the SWCNTs are armchair single wall carbon nanotubes (i.e. (n,n)-SWCNTs), the integer n can vary over a broad range, e.g. from 2 to 30, preferably from 3 to 20, or from 5 to 15.

If the SWCNTs are zig-zag single wall carbon nanotubes (i.e. (n,0)-SWCNTs), the integer n can vary over a broad range, e.g. from 3 to 50, preferably from 4 to 35, or from 5 to 15.

If the SWCNTs are chiral single wall carbon nanotubes (i.e. (n,m)-SWCNTs, wherein n≠m), each of the integers n and m can vary over a broad range, e.g. 5≦n+m≦60; or 6≦n+m≦20.

As indicated above, in step (i) of the process of the present invention, a precursor element of the single wall carbon nanotube is provided, wherein the precursor element comprises a segment S_{SWCNT} of the single wall carbon nanotube, the segment S_{SWCNT} being made of at least one ring formed by ortho-fused benzene rings and having a first end E1 which is open and a second end E2 which is opposite to the first end E1.

This segment S_{SWCNT} can also be referred to as a nanotube belt. If many of these segments S_{SWCNT} were attached to each other via their ends, they would form the predefined single wall carbon nanotube.

The second end E2 of the segment S_{SWCNT} can be open as well. Alternatively, the precursor element further comprises a cap of the single wall carbon nanotube, the cap being attached to the second end E2 of the segment S_{SWCNT} (i.e. thereby resulting in a nanotube segment S_{SWCNT} having an open end E1 and a closed end E2).

The one or more rings the carbon nanotube segment S_{SWCNT} is made of are formed by ortho-fused benzene rings. A system in which any two adjacent aromatic rings have two, and only two, adjacent atoms in common are said to be ‘ortho-fused’. If the first benzene ring and the final benzene ring in a sequence of ortho-fused benzene rings are also fused to each other, a segment ring of the carbon nanotube segment S_{SWCNT} is formed. The fused benzene rings forming the segment ring can all be in a linear sequence (such a ring also referred to as a cyclacene) or an angular sequence (such a ring also referred to as a cyclophenacene), or some of the fused benzene rings forming the segment ring are in a linear sequence whereas other fused benzene rings in the same segment ring are in an angular sequence. The segment S_{SWCNT} may have further segment rings (each segment ring being formed by ortho-fused benzene rings). If two or more segment rings are present and neighbouring segment rings are fused to each, this means that the “upper rim” of the first ring and the “lower rim” of the neighbouring ring are formed by the same carbon atoms. However, it is also possible that the segment S_{SWCNT} is just made of a single segment ring. Furthermore, it is also possible that the segment S_{SWCNT} is not terminated, either at one of its ends E1 or E2 or at both ends, by a complete segment ring, of course under the provision that at least one segment ring formed by ortho-fused benzene rings is present.

Preferably, the segment S_{SWCNT} is made of (i.e. consists of) from 1 to 10 segment rings, each segment ring being formed by ortho-fused benzene rings.

Preferably, the precursor element is prepared from a polycyclic aromatic compound.

As known to the skilled person, a polycyclic aromatic compound is a compound containing fused aromatic rings as a structural element.

In a preferred embodiment, the precursor element is made of the segment S_{SWCNT} and a cap of the SWCNT to be prepared, the cap being attached to the second end E2 of the segment S_{SWCNT} (thereby resulting in a segment S_{SWCNT} having a closed end E2 and an open end E1).

It is known to the skilled person that end caps (or parts thereof) of SWCNTs can be prepared from polycyclic aromatic compounds. Reference can be made to US 2012/0177561 A1 and K. Y. Amsharov, A. Müller, Eur. J. Org. Chem., 2012, pp. 6155-6164. In the present invention, polycyclic aromatic compounds are preferably used which, if subjected to an intramolecular cyclization, do not just provide the end cap itself of the pre-defined SWCNT but an “extended end cap” additionally containing a carbon nanotube segment S_{SWCNT} (i.e. at least one ring formed by ortho-fused benzene rings) attached to the cap.

If the segment S_{SWCNT} has an end E2 which is closed by a SWCNT cap, the number of segment rings can be e.g. from 1 to 10. However, as complexity of the organic synthesis of polycyclic aromatic compounds may increase with increasing molecular size, the number of segment rings is preferably kept low, e.g. 1 to 3 segment rings or 1 to 2 segment rings or even just one segment ring.

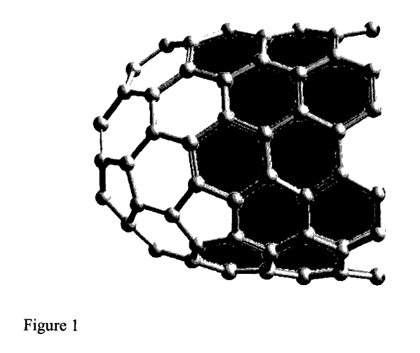

FIG. 1 shows the structure of an exemplary precursor element from which a (6,6) single wall carbon nanotube can be prepared. In this exemplary structure, the precursor element is not only made of a SWCNT segment (gray shading) but additionally contains a cap at one end of the SWCNT segment. The SWCNT segment (gray-shaded area) is made of segment rings, each of these segment rings being formed by ortho-fused benzene rings.

The polycyclic aromatic compound used for preparing the precursor element (which is preferably made of the segment S_{SWCNT} and a SWCNT cap attached to the second end E2 of the segment S_{SWCNT}) can be e.g. a compound of formula (1) having a central benzene ring with residues R¹ to R⁶

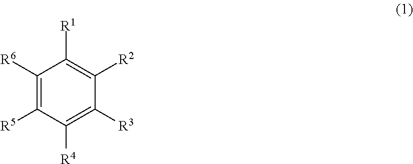

wherein
each of the residues R¹, R³ and R⁵, which can be the same or different, has the following formula (2)

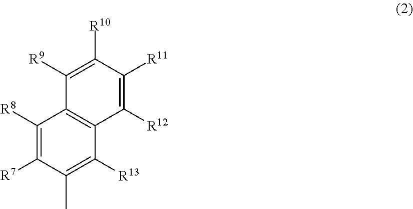

wherein
at least one of the residues R⁷ to R¹³ is a substituted or unsubstituted phenyl or a polycyclic aromatic group; or
at least two of the residues R⁷ to R¹³ which are adjacent to each other form together a monocyclic or polycyclic aromatic group;
   the residues R², R⁴ and R⁶, which can be the same or different, are hydrogen; or have the following formula (3)

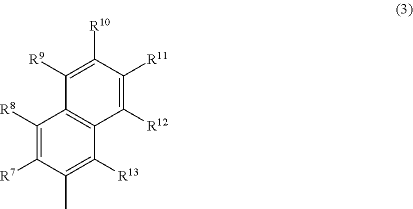

wherein R⁷ to R¹³ have the same m meaning as in formula (2); or
   represent a chemical bond connecting the central benzene ring with the residue R⁷ or R¹³ in formula (2).

As already mentioned above, the residues R¹, R³ and R⁵ can be identical or different from each other. The same is true for the residues R², R⁴ and R⁶. Furthermore, if present, the residues of formula (3) can be identical with or different from the residues of formula (2).

Optionally, if one or more of the residues R², R⁴ and R⁶ are of formula (3), it is possible that R⁷ in one of the residues R¹, R³, and R⁵ of formula (2) and R¹³ in one of the adjacent residues R², R⁴, and R⁶ of formula (3), and/or R¹³ in one of the residues R¹, R³, and R⁵ of formula (2) and R⁷ in one of the adjacent residues R², R⁴, and R⁶ of formula (3) represent/form together a chemical bond.

If one of the residues R⁷ to R¹³ in formula (2) is a substituted phenyl, the one or more substituents can be e.g. a phenyl group. The same is true for the residues R⁷ to R¹³ in formula (3.)

If one of the residues R⁷ to R¹³ in formula (2) is a polycyclic aromatic group, the number of fused benzene rings in said polycyclic aromatic group can vary e.g. from 2 to 8, preferably from 2 to 6 or from 2 to 4. The same is true for the residues R⁷ to R¹³ in formula (3.)

If at least two of the residues R⁷ to R¹³ of formula (2) which are adjacent to each other form together a monocyclic aromatic group, it is preferably a six-membered aromatic ring. The same is true for the residues R⁷ to R¹³ in formula (3.)

If at least two of the residues R⁷ to R¹³ of formula (2) which are adjacent to each other form together a polycyclic aromatic group, the number of fused benzene rings in said polycyclic aromatic group can vary e.g. from 2 to 8, preferably from 2 to 6 or from 2 to 4. The same is true for the residues R⁷ to R¹³ in formula (3.)

If the polycyclic aromatic group is formed by two fused benzene rings, it is derived from naphthalene, which can be substituted or unsubstituted.

If the polycyclic aromatic group is formed by three fused benzene rings, it is derived from phenanthrene or anthracene, each of which can be substituted or unsubstituted.

If the polycyclic aromatic group is formed by four fused benzene rings, it is derived from e.g. chrysene, tetraphene (i.e. benz[a]anthracene), tetracene, triphenylene (i.e. 9,10-benzophenanthrene), benzo[c]phenantrene, or pyrene, each of which can be substituted or unsubstituted.

If the polycyclic aromatic group is formed by five fused benzene rings, it is derived from e.g. dibenzo-anthracenes such as dibenzo[a,h]anthracene, dibenzo[a,c]anthracene, 1,2:7,8-dibenzanthracene (also known as dibenz[aj]anthracene and 1,2:5,6-dibenzoanthracene; benzo-chrysenes such as picene (also known as benzo[a]chrysene) and benzo[b]chrysene; pentacene; dibenzo-phenanthrenes such as 1,2:3,4-dibenzophenanthrene and 3,4,5,6-dibenzophenantrene; benzo-naphthacenes such as benzo[a]naphthacene; benzo-pyrenes such as benzo[a]pyrene and benzo[e]pyrene; each of which can be substituted or unsubstituted.

If the polycyclic aromatic group is formed by six fused benzene rings, it is derived from e.g. benzo-picenes such as benzo[s]picene; benzo-pentacenes; benzo-perylenes; or dibenzo-pyrenes; each of which can be substituted or unsubstituted.

Alternatively, the polycyclic aromatic compound used for preparing the precursor element (which is preferably made of the segment S_{SWCNT} and a SWCNT cap attached to the second end E2 of the segment S_{SWCNT}) can be e.g. a compound of formula (4)

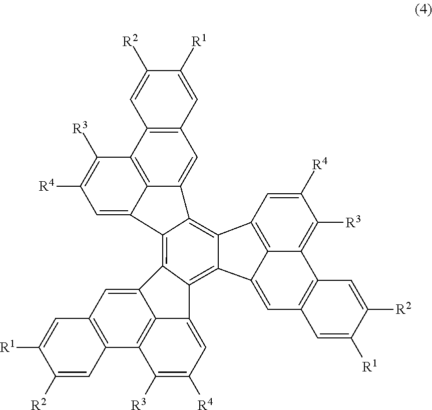

wherein
at least one of the residues R¹ to R⁴ is a substituted or unsubstituted phenyl or a polycyclic aromatic group; or
the residues R¹ and R² and/or the residues R³ and R⁴ form together a monocyclic or polycyclic aromatic group.

With regard to exemplary polycyclic aromatic groups, reference can be made to the statements provided above.

If one or more of the residues R¹ to R⁴ is/are a substituted phenyl, the one or more substituents can be e.g. phenyl.

In formula (4), the residues R¹ and R², independently from each other, can be e.g. hydrogen, a substituted or unsubstituted phenyl or a polycyclic aromatic group; and the residues R³ and R⁴ may form together a benzene ring carrying a phenyl or biphenyl substituent, or the residues R³ and R⁴ may form together a polycyclic aromatic group of 2 to 6 fused benzene rings, which can be substituted or unsubstituted.

Alternatively, the polycyclic aromatic compound used for preparing the precursor element (which is preferably made of the segment S_{SWCNT} and a SWCNT cap attached to the second end E2 of the segment S_{SWCNT}) can be e.g. a corannulene compound.

An exemplary polycyclic aromatic compound that can be used for preparing (6,6) armchair SWCNTs has one of the following formulas (I) and (II):

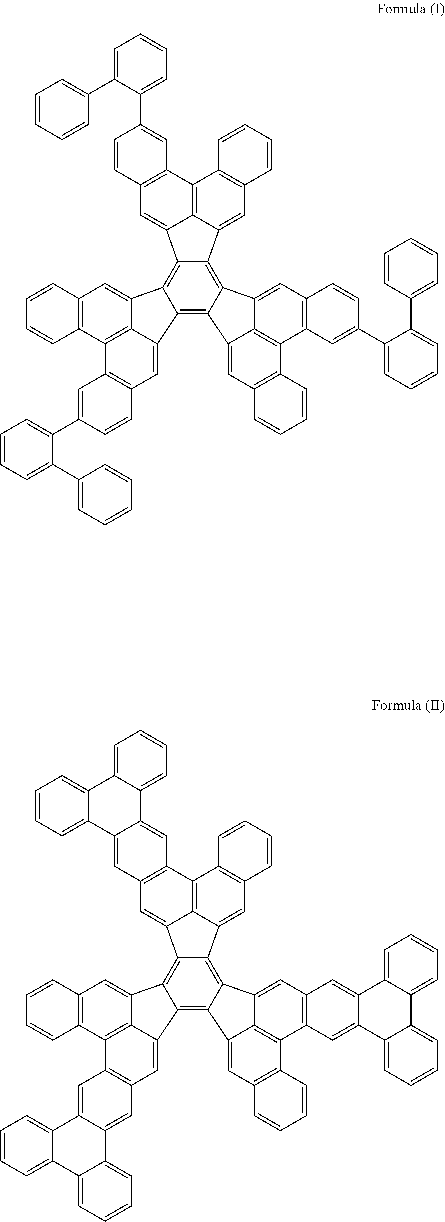

An exemplary polycyclic aromatic compound that can be used for preparing (9,9) armchair SWCNTs has the following formula (III):

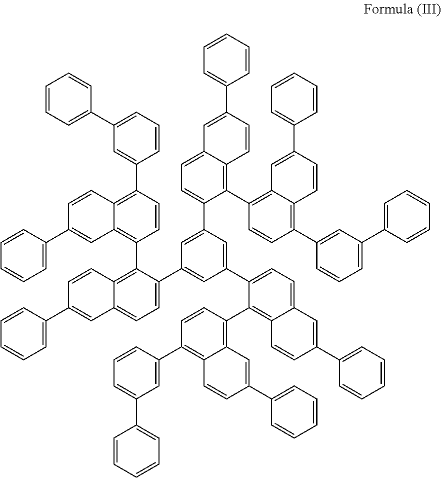 [SMILES: c1ccc(-c2cccc(-c3ccc(-c4c(-c5cc(-c6ccc7cc(-c8ccccc8)ccc7c6-c6ccc(-c7cccc(-c8ccccc8)c7)c7ccc(-c8ccccc8)cc67)cc(-c6ccc7cc(-c8ccccc8)ccc7c6-c6ccc(-c7cccc(-c8ccccc8)c7)c7ccc(-c8ccccc8)cc67)c5)ccc5cc(-c6ccccc6)ccc45)c4cc(-c5ccccc5)ccc34)c2)cc1]

An exemplary compound that can be used for preparing (9,0) zig-zag SWCNTs has the following formula (IV):

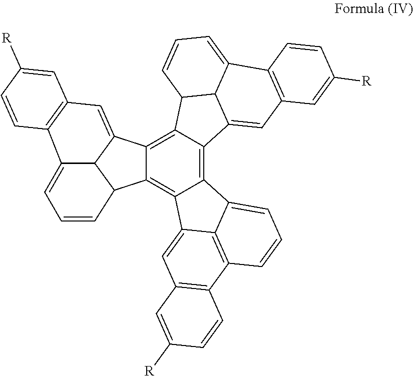

wherein R is phenyl (i.e. —C₆H₅).

An exemplary compound that can be used for preparing (12,6) chiral SWCNTs has one of the following formulas (V) and (VI):

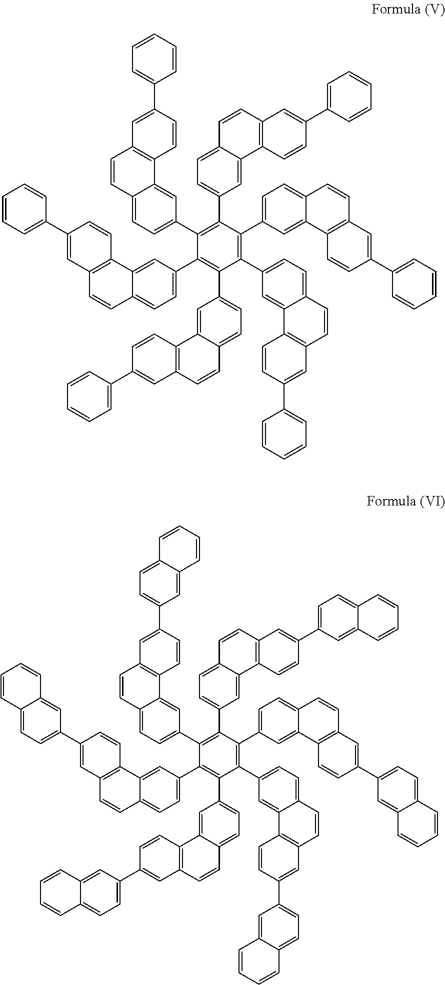

An exemplary compound that can be used for preparing (12,0) zig-zag SWCNTs has one of the following formulas (VII) and (VIII):

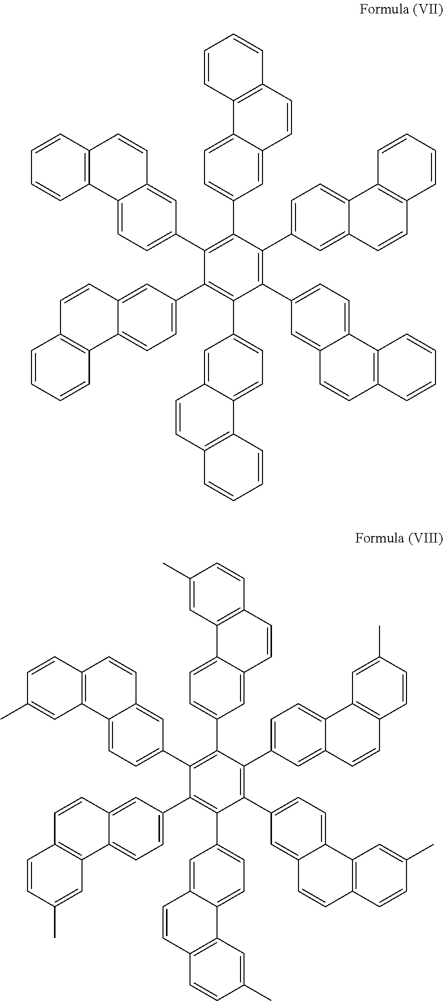

An exemplary compound that can be used for preparing (12,3) chiral SWCNTs has the following formula (IX):

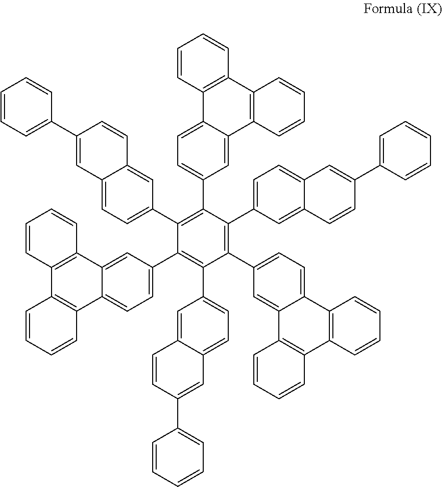 [SMILES: c1ccc(-c2ccc3cc(-c4c(-c5ccc6c7ccccc7c7ccccc7c6c5)c(-c5ccc6cc(-c7ccccc7)ccc6c5)c(-c5ccc6c7ccccc7c7ccccc7c6c5)c(-c5ccc6cc(-c7ccccc7)ccc6c5)c4-c4ccc5c6ccccc6c6ccccc6c5c4)ccc3c2)cc1]

An exemplary compound that can be used for preparing (12,1) chiral SWCNTs has the following formula (X):

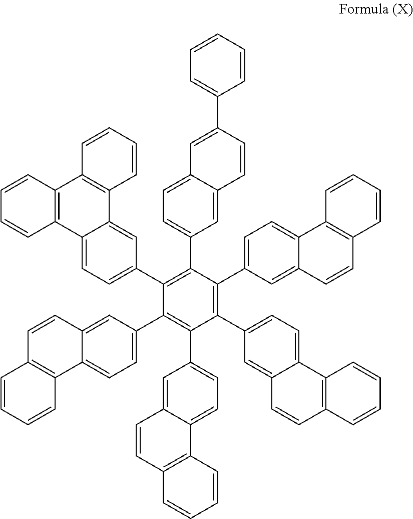 [SMILES: c1ccc(-c2ccc3cc(-c4c(-c5ccc6c(ccc7ccccc76)c5)c(-c5ccc6c(ccc7ccccc76)c5)c(-c5ccc6c(ccc7ccccc76)c5)c(-c5ccc6c(ccc7ccccc76)c5)c4-c4ccc5c6ccccc6c6ccccc6c5c4)ccc3c2)cc1]

An exemplary compound that can be used for preparing (11,3) chiral SWCNTs has the following formula (XI):

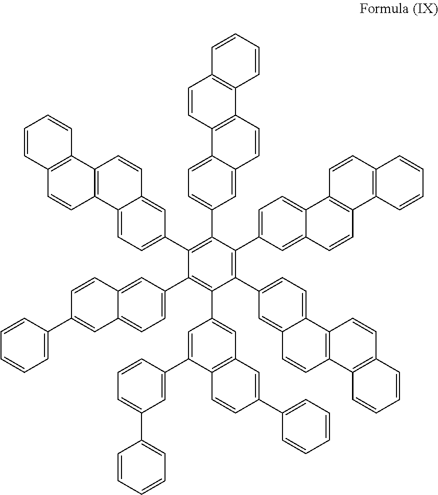 [SMILES: c1ccc(-c2cccc(-c3cc(-c4c(-c5ccc6cc(-c7ccccc7)ccc6c5)c(-c5ccc6c(ccc7c8ccccc8ccc67)c5)c(-c5ccc6c(ccc7c8ccccc8ccc67)c5)c(-c5ccc6c(ccc7c8ccccc8ccc67)c5)c4-c4ccc5c(ccc6c7ccccc7ccc56)c4)cc4cc(-c5ccccc5)ccc34)c2)cc1]

An exemplary compound that can be used for preparing (12,2) chiral SWCNTs has the following formula (XII):

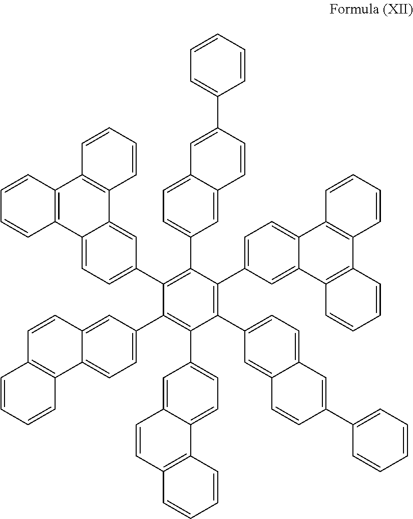 [SMILES: c1ccc(-c2ccc3cc(-c4c(-c5ccc6c(ccc7ccccc76)c5)c(-c5ccc6c(ccc7ccccc76)c5)c(-c5ccc6c7ccccc7c7ccccc7c6c5)c(-c5ccc6cc(-c7ccccc7)ccc6c5)c4-c4ccc5c6ccccc6c6ccccc6c5c4)ccc3c2)cc1]

An exemplary compound that can be used for preparing (10,6) chiral SWCNTs has the following formula (XIII):

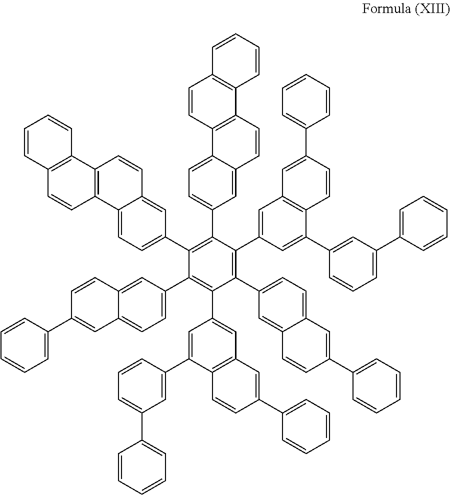 [SMILES: c1ccc(-c2cccc(-c3cc(-c4c(-c5ccc6cc(-c7ccccc7)ccc6c5)c(-c5cc(-c6cccc(-c7ccccc7)c6)c6ccc(-c7ccccc7)cc6c5)c(-c5ccc6c(ccc7c8ccccc8ccc67)c5)c(-c5ccc6c(ccc7c8ccccc8ccc67)c5)c4-c4ccc5cc(-c6ccccc6)ccc5c4)cc4cc(-c5ccccc5)ccc34)c2)cc1]

An exemplary compound that can be used for preparing (11,5) chiral SWCNTs has the following formula (XIV):

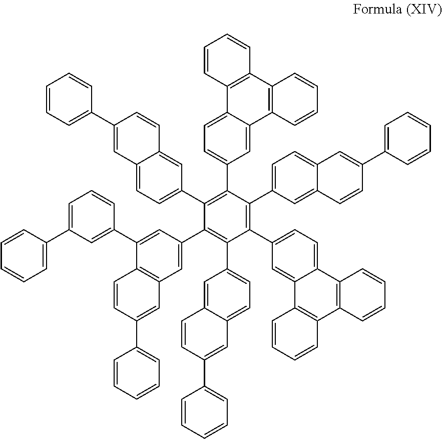 [SMILES: c1ccc(-c2cccc(-c3cc(-c4c(-c5ccc6cc(-c7ccccc7)ccc6c5)c(-c5ccc6c7ccccc7c7ccccc7c6c5)c(-c5ccc6cc(-c7ccccc7)ccc6c5)c(-c5ccc6c7ccccc7c7ccccc7c6c5)c4-c4ccc5cc(-c6ccccc6)ccc5c4)cc4cc(-c5ccccc5)ccc34)c2)cc1]

An exemplary compound that can be used for preparing (10,7) chiral SWCNTs has the following formula (XV):

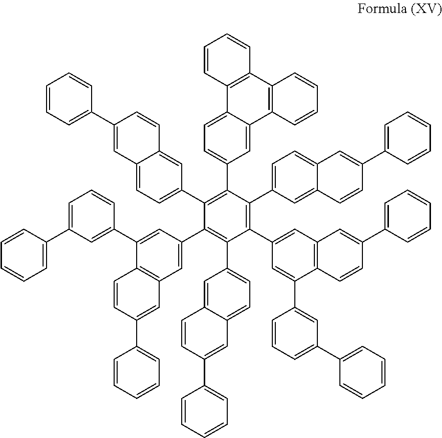 [SMILES: c1ccc(-c2cccc(-c3cc(-c4c(-c5ccc6cc(-c7ccccc7)ccc6c5)c(-c5cc(-c6cccc(-c7ccccc7)c6)c6ccc(-c7ccccc7)cc6c5)c(-c5ccc6cc(-c7ccccc7)ccc6c5)c(-c5ccc6c7ccccc7c7ccccc7c6c5)c4-c4ccc5cc(-c6ccccc6)ccc5c4)cc4cc(-c5ccccc5)ccc34)c2)cc1]

An exemplary compound that can be used for preparing (11,4) chiral SWCNTs has the following formula (XVI):

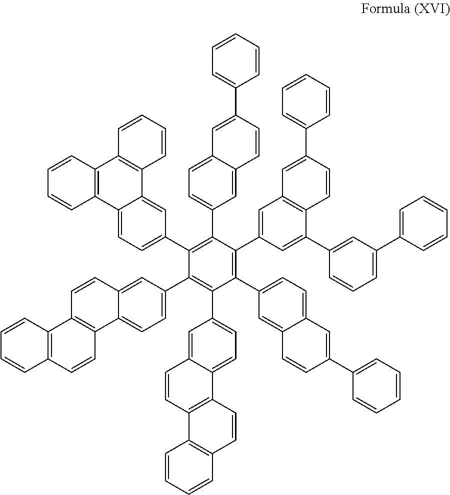 [SMILES: c1ccc(-c2cccc(-c3cc(-c4c(-c5ccc6cc(-c7ccccc7)ccc6c5)c(-c5ccc6c(ccc7c8ccccc8ccc67)c5)c(-c5ccc6c(ccc7c8ccccc8ccc67)c5)c(-c5ccc6c7ccccc7c7ccccc7c6c5)c4-c4ccc5cc(-c6ccccc6)ccc5c4)cc4cc(-c5ccccc5)ccc34)c2)cc1]

An exemplary compound that can be used for preparing (8,2) chiral SWCNTs has the following formula (XVII):

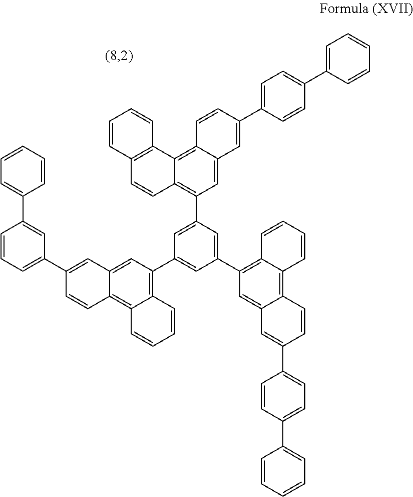 [SMILES: c1ccc(-c2ccc(-c3ccc4c(c3)cc(-c3cc(-c5cc6cc(-c7cccc(-c8ccccc8)c7)ccc6c6ccccc56)cc(-c5cc6cc(-c7ccc(-c8ccccc8)cc7)ccc6c6c5ccc5ccccc56)c3)c3ccccc34)cc2)cc1]

An exemplary compound that can be used for preparing (7,4) chiral SWCNTs has the following formula (XIII):

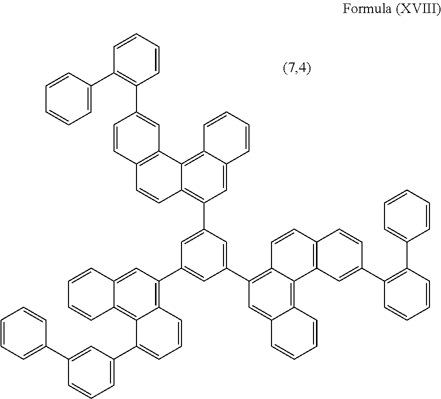 [SMILES: c1ccc(-c2cccc(-c3cccc4c(-c5cc(-c6cc7ccccc7c7c6ccc6ccc(-c8ccccc8-c8ccccc8)cc67)cc(-c6cc7ccccc7c7c6ccc6ccc(-c8ccccc8-c8ccccc8)cc67)c5)cc5ccccc5c34)c2)cc1]

An exemplary compound that can be used for preparing (11,0) zig-zag SWCNTs has the following formula (XIX):

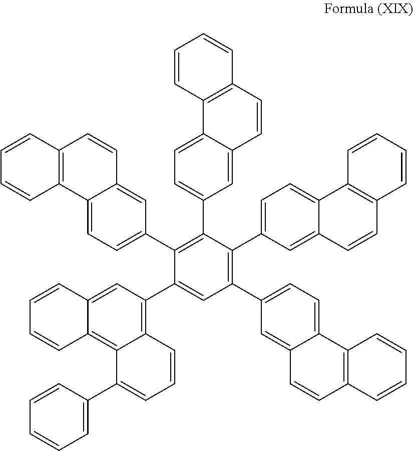 [SMILES: c1ccc(-c2cccc3c(-c4cc(-c5ccc6c(ccc7ccccc76)c5)c(-c5ccc6c(ccc7ccccc76)c5)c(-c5ccc6c(ccc7ccccc76)c5)c4-c4ccc5c(ccc6ccccc65)c4)cc4ccccc4c23)cc1]

An exemplary compound that can be used for preparing (10,2) chiral SWCNTs has the following formula (XX):

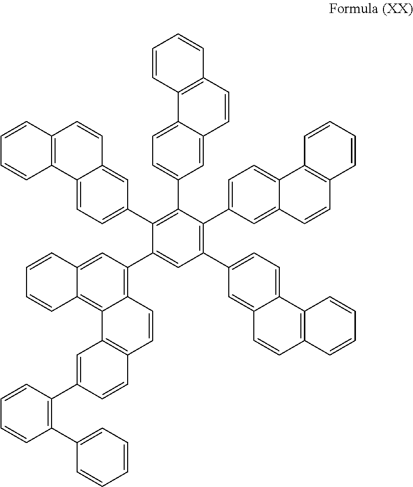 [SMILES: c1ccc(-c2ccccc2-c2ccc3ccc4c(-c5cc(-c6ccc7c(ccc8ccccc87)c6)c(-c6ccc7c(ccc8ccccc87)c6)c(-c6ccc7c(ccc8ccccc87)c6)c5-c5ccc6c(ccc7ccccc76)c5)cc5ccccc5c4c3c2)cc1]

An exemplary compound that can be used for preparing (7,7) armchair SWCNTs has the following formula (XXI):

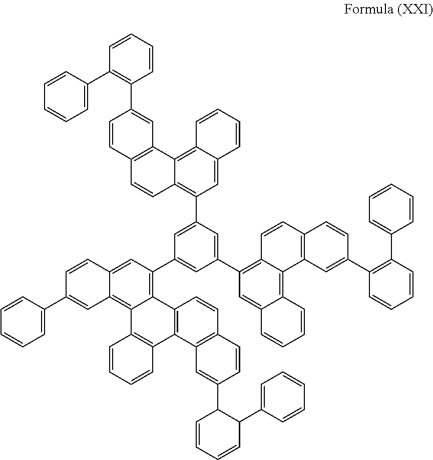 [SMILES: C1=CC(c2ccccc2)C(c2ccc3ccc4c5c(-c6cc(-c7cc8ccccc8c8c7ccc7ccc(-c9ccccc9-c9ccccc9)cc78)cc(-c7cc8ccccc8c8c7ccc7ccc(-c9ccccc9-c9ccccc9)cc78)c6)cc6ccc(-c7ccccc7)cc6c5c5ccccc5c4c3c2)C=C1]

An exemplary compound that can be used for preparing (8,8) armchair SWCNTs has the following formula (XXII):

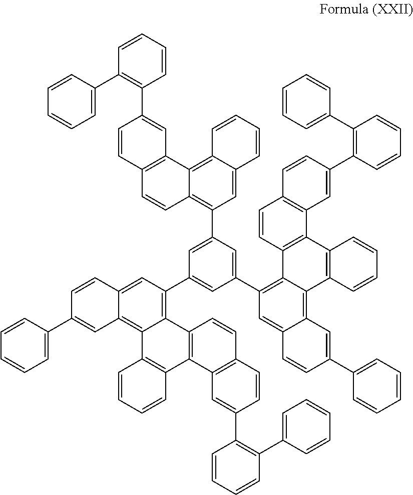 [SMILES: c1ccc(-c2ccc3cc(-c4cc(-c5cc6ccccc6c6c5ccc5ccc(-c7ccccc7-c7ccccc7)cc56)cc(-c5cc6ccc(-c7ccccc7)cc6c6c7ccccc7c7c8cc(-c9ccccc9-c9ccccc9)ccc8ccc7c56)c4)c4c5ccc6ccc(-c7ccccc7-c7ccccc7)cc6c5c5ccccc5c4c3c2)cc1]

An exemplary compound that can be used for preparing (9,5) armchair SWCNTs has the following formula (XXIII):

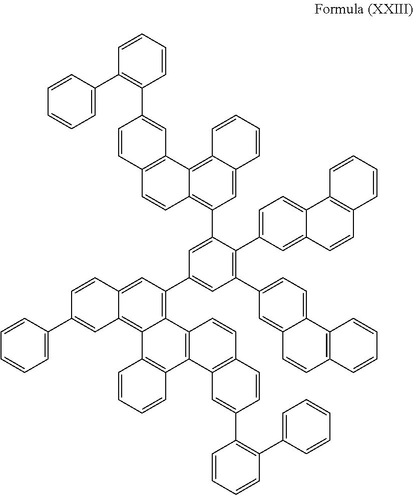 [SMILES: c1ccc(-c2ccc3cc(-c4cc(-c5ccc6c(ccc7ccccc76)c5)c(-c5ccc6c(ccc7ccccc76)c5)c(-c5cc6ccccc6c6c5ccc5ccc(-c7ccccc7-c7ccccc7)cc56)c4)c4c5ccc6ccc(-c7ccccc7-c7ccccc7)cc6c5c5ccccc5c4c3c2)cc1]

In any of these compounds (I) to (XXIII), at least some of the hydrogen atoms can be substituted by halide atoms (such as Cl, Br, or I).

If subjected to an intramolecular cyclization, each of the polycyclic aromatic compounds (I) to (XXIII) forms a precursor element which consists of a SWCNT segment S_{SWCNT} and a SWCNT cap attached to one end of the segment. As discussed above, the segment S_{SWCNT} of each of the precursor elements is made of at least one ring formed by ortho-fused benzene rings and has an open end E1.

If a polycyclic aromatic compound is used for providing the precursor element, said polycyclic aromatic compound can be prepared by organic synthesis procedures principally known to the skilled person.

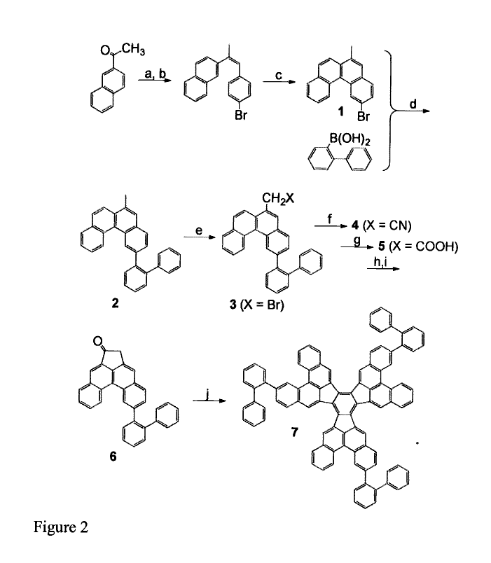

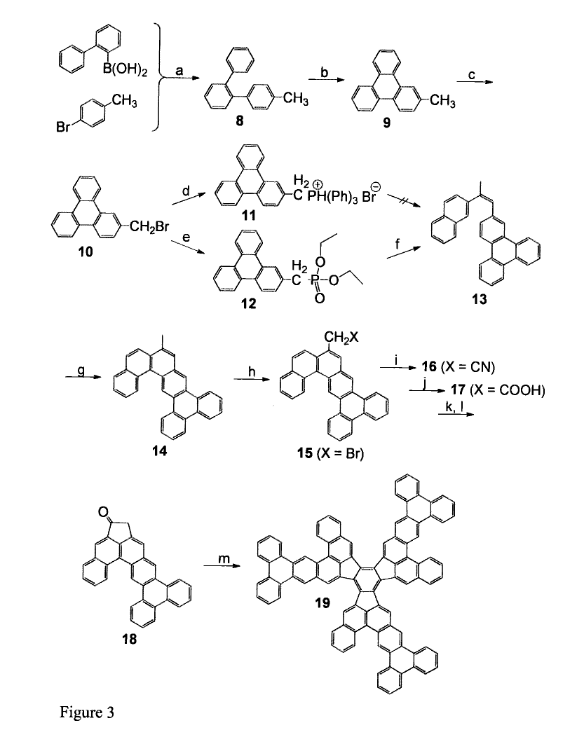

Just as an example, the synthetic routes to the polycyclic aromatic compounds (I) and (II) are illustrated in FIGS. 2 and 3.

The synthetic route shown in FIG. 2 for preparing the polycyclic aromatic compound of formula (I) (in FIG. 2: compound 7) includes the following steps:

a) PPh₃, toluene, reflux, 95%; b) BrPh₃PCH₂PhBr, KOtBu, EtOH, reflux, 81%; c) I₂, hv, propylene oxide, cyclohexane, 72%; d) Pd(PPh₃)₄, Cs₂CO₃, toluene/MeOH, 110° C., 79%; e) NBS, DBPO, CCl₄, reflux, 70%; f) NaCN, DMSO, RT, 40%; g) H₂SO₄, H₂O, HOAc, reflux, 98%; h) SOCl₂, 65° C.; i) AlCl₃, CH₂Cl₂, RT, 57%; j) propanoic acid, TsOH, o-DCB, 180° C., 65%.

The synthetic route shown in FIG. 3 for preparing the polycyclic aromatic compound of formula (II) (in FIG. 3: compound 19) includes the following steps: a) Pd(PPh₃)₄, K₂CO₃, toluene/MeOH, 110° C., 86%; b) I₂, hv, propylene oxide, cyclohexane, 52%; c) NBS, DBPO, CCl₄, reflux, 87%; d) PPh₃, toluene, reflux, 71%; e) P(OEt)₃, 160° C., 60%; f) C₁₀H₇COCH₃, KOtBu, THF, 50° C., 31%; g) I₂, hv, propylene oxide, cyclohexane, 24%; h) NBS, DBPO, CCl₄, reflux, 60%; i) NaCN, DMSO, RT, 75%; j) H₂SO₄, H₂O, HOAc, reflux, 73%; k) SOCl₂, 65° C.; 1) AlCl₃, CH₂Cl₂, RT, 36%; m) TiCl₄, o-DCB, 180° C., 35%.

The precursor element can be prepared from the polycyclic aromatic compound by known methods such as intramolecular cyclization. Preferred intramolecular cyclization reactions are cyclodehydrogenation, cyclodehalogenation and Bergman cyclization.

When the polycyclic aromatic compound is subjected to a conversion step such as cyclodehydrogenation, aromatic rings of the polycyclic aromatic compound get connected via an intramolecular reaction and the aromatic compound, which can typically be considered as a more or less flat molecule, is converted to a “bowl-like” SWCNT precursor element made of the tube segment S_{SWCNT} and the SWCNT cap closing one end of the tube segment while the other end of the tube segment remains open.

If an intramolecular cyclization such as cyclodehydrogenation is used for converting the polycyclic aromatic compound to the precursor element, it is preferably a surface-assisted (also referred to as “surface-catalyzed”) intramolecular cyclization. Appropriate process conditions and surfaces on which the polycyclic aromatic compound can be subjected to an intramolecular cyclization such as cyclodehydrogenation are known to the skilled person. In this context, reference can be made e.g. to K. Amsharov et al., Angew. Chem. Int. Ed. 2010, pp. 9392-9396 and G. Otero et al., Nature, Vol. 454, 2008, pp. 865-868.

In a preferred embodiment, the surface-assisted intramolecular cyclization is carried out on the surface of a metal-containing catalyst, more specifically on a metal surface of the metal-containing catalyst. Preferably, the metal is Pd, Pt, Ru, Ir, Rh, Au, Ag, Fe, Co, Cu, Ni, or any mixture or alloy thereof.

The polycyclic aromatic compound can be deposited on the catalyst surface by any process suitable for deposition of organic compounds on a surface. The process may e.g. be a vacuum deposition (sublimation) process, a solution based process such as spin coating, spray coating, dip coating, printing, electrospray deposition, pulsed jet deposition, dry contact transfer or dry imprint, a laser induced desorption process, or preparative mass spectrometry (especially for deposition of compounds which are only available in impure mixtures).

If the polycyclic aromatic compound comprises groups that may rotate about a single bond (e.g. a phenyl or biphenyl residue at the outer periphery of the molecule) and therefore shows conformational isomerism, the molecules deposited on the catalyst surface may have different conformations. However, exclusively those molecules that are adsorbed on the catalyst surface in an appropriate conformation will form (e.g. by cyclodehydrogenation) the SWCNT precursor element, which in turn will then grow to the final SWCNT in the vapour phase reaction of step (ii). Any molecule adsorbed in an inappropriate conformation will not form a SWCNT precursor element that may subsequently grow in step (ii). So, only those SWCNTs having the pre-defined chirality will actually be formed in step (ii). In other words, with the process of the present invention, SWCNTs of pre-defined chirality can be obtained in extremely high purity (i.e. purity of about 100%).

Preferably, the polycyclic aromatic compounds are deposited under conditions so as to achieve a surface density (i.e. number of compounds deposited per unit area of catalyst surface) which is high enough for optimizing yield of precursor elements and, consequently, SWCNTs, but low enough for suppressing contact between neighbouring compounds as much as possible. If two or more neighbouring polycyclic aromatic compounds come into contact, the yield of defined precursor elements obtained after the conversion step (i) may decrease.

For maximizing the contact area between the polycyclic aromatic compound and the catalyst surface and thereby improving efficiency of the cyclodehydrogenation step, the average diameter of the metal-containing catalyst particles should be kept sufficiently large.

Preferably, the metal-containing catalyst of step (i) is in the form of particles having an average diameter d_{cat} satisfying the following relation: d_{cat}>2×d_{SWCNT}, more preferably: d_{cat}>4×d_{SWCNT}; even more preferably: d_{cat}>10×d_{SWCNT} or d_{cat}>20×d_{SWCNT} or d_{cat}>50×d_{SWCNT}, or in the form of a continuous film.

The particles of the metal-containing catalyst in step (i) may have an average particle size of e.g. at least 5 nm, more preferably at least 10 nm, even more preferably at least 20 nm or at least 50 nm or at least 100 nm, under the provision that d_{cat}>2×d_{SWCNT}.

The average particle size can be determined by an image analysis (e.g. images taken from transmission electron microscopy (TEM) or scanning electron microscopy (SEM)). In this image-based particle size analysis, the size distribution of the particles shown on the TEM (or SEM) image is determined. The particle size of an individual particle is its largest extension as available from the image. The peak value of the size distribution curve is the average particle size.

The term “catalyst in the form of a continuous film” means that the dimensions of the surface provided by the catalyst and on which the polycyclic aromatic compounds are deposited, are by far exceeding the molecular size of the aromatic compound. Preferably, such a catalyst in the form of a film provides a flat surface, thereby improving interaction between the polycyclic aromatic compound and the catalyst surface and increasing efficiency of the intramolecular cyclization.

The catalyst used in step (i) can be crystalline, either partly or completely. However, it is also possible that the catalyst in step (i) is amorphous.

Catalysts that can be used in step (i) for intramolecular cyclization, are generally known to the skilled person and are commercially available or can be prepared by commonly known standard methods.

As known to the skilled person, the diameter of a SWCNT can be calculated from the following equation:

d_{SWCNT}=(n²+m²+nm)^{1/2}×0.0783 nm

wherein n and m are the integers defining the chiral vector and therefore the structure of the SWCNT. Furthermore, as already discussed above, by selecting an appropriate polycyclic aromatic compound, a specific SWCNT with characteristic n,m-integers is obtained. From these known integers n and m, the diameter of the SWCNT can be calculated.

Appropriate process conditions for converting the polycyclic aromatic compound to the SWCNT precursor element in step (i) are principally known to the skilled person.

The temperature at which the intramolecular cyclization is carried out may vary over a broad range. Preferably, step (i) is carried out at a temperature T₁ of from 100° C. to 1000° C., more preferably of from 200 to 800° C. or from 200 to 600° C.

Alternatively, instead of using a nanotube segment S_{SWCNT} which has an open end E1 and a closed end E2, it is also possible to use a nanotube segment S_{SWCNT} which is open at both ends.

Such short nanotube segments which are open at both ends can be prepared chemically, e.g. via chemical reactions described in Pure Appl. Chem., 2012, Vol. 84, No. 4, pp. 907-916. Alternatively, a SWCNT be cut into smaller segments, e.g. electron beam lithography and oxygen plasma ion etching (see e.g. Nano Letters, 2009, Vol. 9, No. 4, pp. 1673-1677).

As indicated above, the process of the present invention comprises a step (ii) which includes growing the precursor element by vapour phase reaction with a carbon-source compound on the surface, preferably the metal surface, of a metal-containing catalyst, wherein the precursor element is in contact with the surface of the metal-containing catalyst via the open end E1 of the segment S_{SWCNT}, and the metal-containing catalyst is in the form of particles having an average diameter d_{cat} satisfying the following relation: d_{cat}>2×d_{SWCNT} or in the form of a continuous film.

The carbon-source compound (i.e. a compound containing one or more carbon atoms) can be selected from those compounds which are typically used for manufacturing carbon nanotubes by chemical vapour deposition (CVD). Such carbon-source compounds are generally known to the skilled person.

A carbon-source compound that can be used in step (ii) of the present invention is e.g. an alkane such as a C₁₋₄ alkane (preferably methane or ethane), an alkene such as ethylene, an alkyne such as acetylene, an alcohol such as a C₁₋₄ alcohol (preferably methanol or ethanol), an aromatic compound such as benzene, carbon monoxide, nitrogen-containing organic compounds (e.g. amines), boron-containing organic compounds, or any mixture thereof. The carbon-source compound can be mixed with heteroatom-containing non-carbon compounds (e.g. NH₃, . . . etc.) which may dope the SWCNT with heteroatoms.

In addition to the carbon-source compound, one may use one or more support gases such as H₂ or H₂O. The use of such support gases in a manufacturing process of carbon nanotubes via vapour phase reaction is generally known to the skilled person.

As a metal of the metal-containing catalyst, those can be mentioned which are typically used for manufacturing carbon nanotubes by chemical vapour deposition (CVD). Preferably, the metal is Pd, Pt, Ru, Ir, Rh, Au, Ag, Fe, Co, Cu, Ni, or any mixture or alloy thereof.

The SWCNT precursor element prepared in step (i) can be brought into contact and deposited on the surface of the metal-containing catalyst by methods commonly known to the skilled person. If a metal-containing catalyst is used in step (i) and is different from the metal-containing catalyst of step (ii), the precursor elements can be transferred to the surface of the metal-containing catalyst of step (ii) by commonly known methods. However, as will be discussed below in further detail, it is preferred to use the same metal-containing catalyst in steps (i) and (ii). In this preferred embodiment, no transfer of the SWCNT precursor elements from the first catalyst to the second catalyst is needed. In other words, in this preferred embodiment, step (ii) includes growing the precursor element by vapour phase reaction with the carbon-source compound on the surface of the metal-containing catalyst of step (i). Preferably, the metal-containing catalyst of step (ii) is in the form of particles having an average diameter d_{cat} satisfying the following relation: d_{cat}>4×d_{SWCNT}, more preferably: d_{cat}>10×d_{SWCNT} or d_{cat}>20×d_{SWCNT} or d_{cat}>50×d_{SWCNT}.

As mentioned above, it is known to the skilled person that the diameter of a SWCNT can be calculated from the following equation:

d_{SWCNT}=(n²+m²+nm)^{1/2}×0.0783 nm

wherein n and m are the integers defining the chiral vector and therefore the structure of the SWCNT. Furthermore, by selecting an appropriate polycyclic aromatic compound, a specific SWCNT with characteristic n,m-integers is obtained. From these known integers n and m, the diameter of the SWCNT can be calculated.

The particles of the metal-containing catalyst in step (ii) may have an average particle size of e.g. at least 5 nm, more preferably at least 10 nm, even more preferably at least 20 nm or at least 50 nm or at least 100 nn, under the provision that d_{cat}>2×d_{SWCNT}.

The average particle size can be determined by an image analysis (e.g. images taken from transmission electron microscopy (TEM) or scanning electron microscopy (SEM)). In this image-based particle size analysis, the size distribution of the particles shown on the TEM (or SEM) image is determined. The particle size of an individual particle is its largest extension as available from the image. The peak value of the size distribution curve is the average particle size.

The term “catalyst in the form of a film” means that the dimensions of the surface provided by the catalyst and on which the polycyclic aromatic compounds are deposited, are by far exceeding the molecular size of the aromatic compound. Preferably, such a catalyst in the form of a film provides a flat surface, thereby improving interaction between the polycyclic aromatic compound and the catalyst surface and increasing efficiency of the intramolecular cyclization.

The catalyst used in step (ii) can be crystalline, either partly or completely. However, it is also possible that the catalyst in step (ii) is amorphous.

In a preferred embodiment, a metal-containing catalyst is also used in step (i), and the metal-containing catalyst of step (i) and the metal-containing catalyst of step (ii) are the same.

The temperature T₂ of step (ii) can vary over a broad range, e.g. from 100 to 1000° C.

In the present invention, it was surprisingly realized that yield of SWCNTs can even be further improved when step (ii) is carried out a low temperature. So, in a preferred embodiment, step (ii) is carried out at a temperature of 700° C. or less, more preferably 600° C. or less, even more preferably 550° C. or less. Appropriate temperature ranges are e.g. from 300 to 700° C., more preferably 350 to 600° C., even more preferably 350 to 550° C.

Preferably, the temperature of step (ii) is kept low enough so as to avoid any spontaneous formation of CNTs in step (ii). In other words, the temperature of step (ii) is preferably low enough so that the carbon-source compound is just initiating growth of the SWCNT precursor elements already provided in step (i) but does not generate further CNTs on the catalyst surface as these further CNTs would not have a well-defined uniform chirality. If the catalyst particles of step (ii) are quite large, this might already be sufficient for suppressing formation of further CNTs in step (ii). If smaller catalyst particles (but still complying with the relation d_{cat}>2×d_{SWCNT}) are used in step (ii), there might be a remaining risk of spontaneously forming CNTs of non-uniform chirality. However, as the SWCNT precursor elements used in the present invention can react with the carbon-source compound even at low temperature, step (ii) can be carried at a temperature which is low enough for suppressing spontaneous formation of further CNTs but high enough for initiating growth of the SWCNT precursor elements provided in step (i).

The pressure at which the vapour phase reaction (and therefore SWCNT growth) are carried out in step (ii) can be varied over a broad range. The vapour phase reaction can be carried out at quite low pressure such as less than 10⁻⁴ mbar or less than 10⁻⁵ mbar or even less than 10⁻⁶ mbar but still provides a sufficient growth rate. If working at low pressure, purity of the final SWCNT might be improved. However, it is also possible to carry out the vapour phase reaction at higher pressure while still obtaining SWCNTs of high quality and extremely high isomeric purity.

In principle, step (ii) can be continued until the SWCNTs have grown to a desired length. Furthermore, if needed, the SWCNT end caps can be removed at a later stage so as to obtain open-ended SWCNTs.

If needed, the SWCNTs of the present invention can be separated from the surface of the metal-containing catalyst by methods commonly known to the skilled person.

According to a further aspect, the present invention provides single wall carbon nanotubes, obtainable by the process described above.

As mentioned above, SWCNTs of uniform structure or chirality (i.e. either armchair or zig-zag or chiral) can be obtained in very high purity by the process of the present invention. Thus, in a preferred embodiment, the single wall carbon nanotubes have an isomeric purity of at least 90%, more preferably at least 95% or even at least 98%, most preferably 100%. An isomeric purity of e.g. 95% means that 95 out of 100 SWCNTs are characterized by the same integers n and m.

As a consequence of the preparation process, the single wall carbon nanotubes of the present invention can be provided on the surface of a metal-containing substrate, which corresponds to the metal-containing catalyst described above and is in the form of a continuous film or in the form of particles having an average diameter d_{substrate} satisfying the following relation: d_{substrate}>2×d_{SWCNT}.

Some of the polycyclic aromatic compounds that can be used for preparing the precursor elements of the present invention have not yet been described in the prior art. So, according to a further aspect, the present invention provides a polycyclic aromatic compound having one of the Formulas (I) to (XXIII):

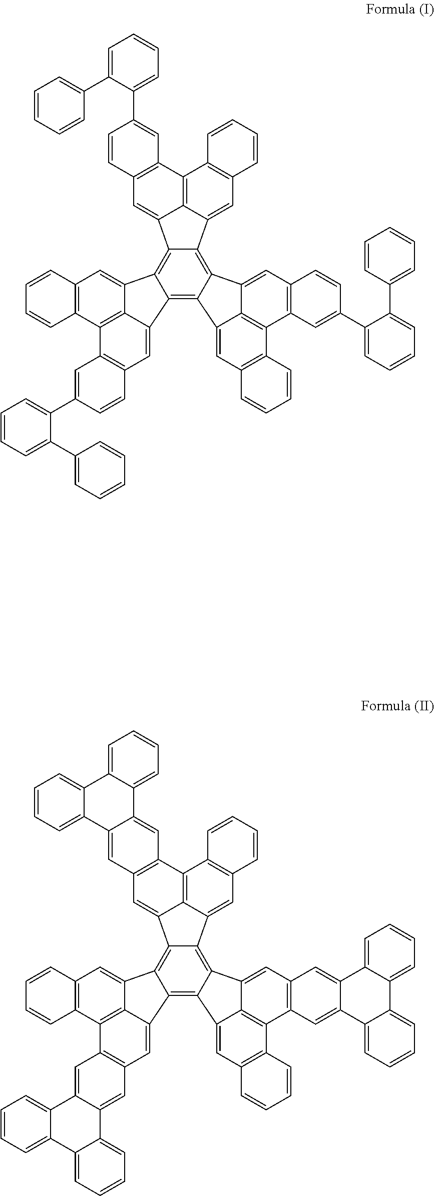

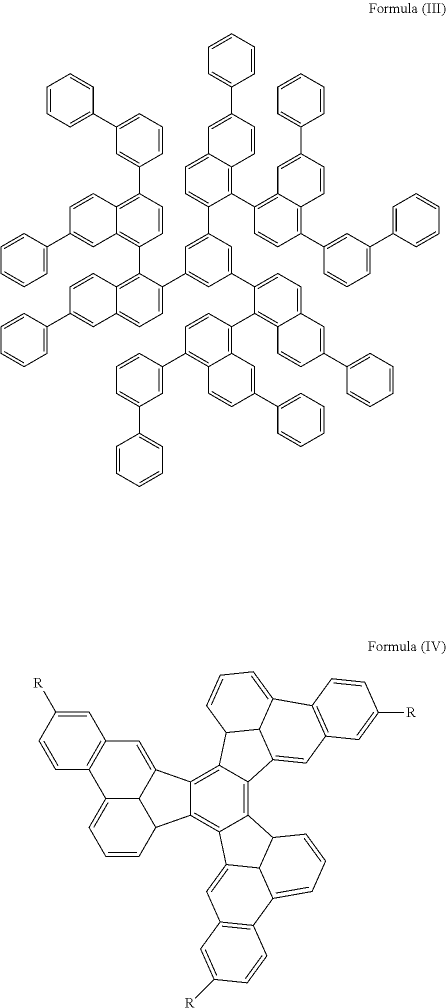

wherein R is phenyl (i.e. —C₆H₅);

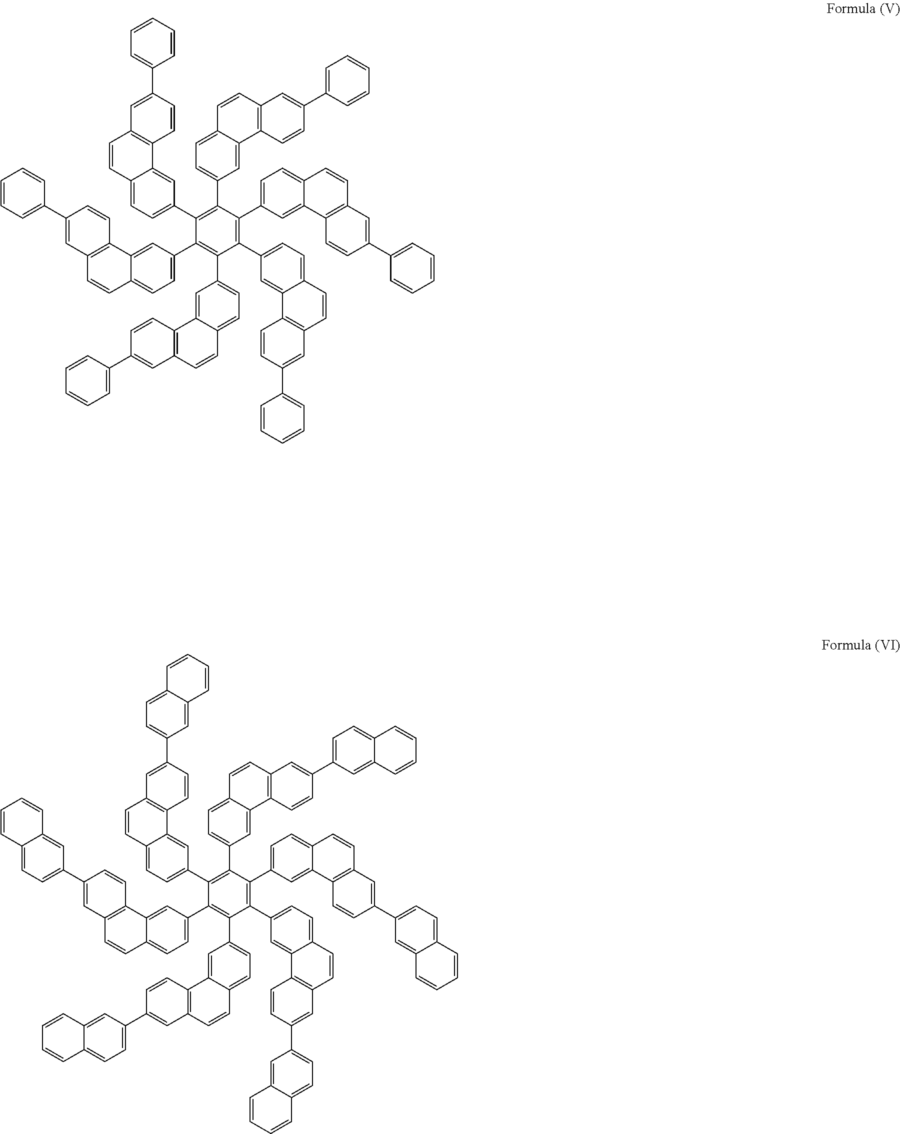

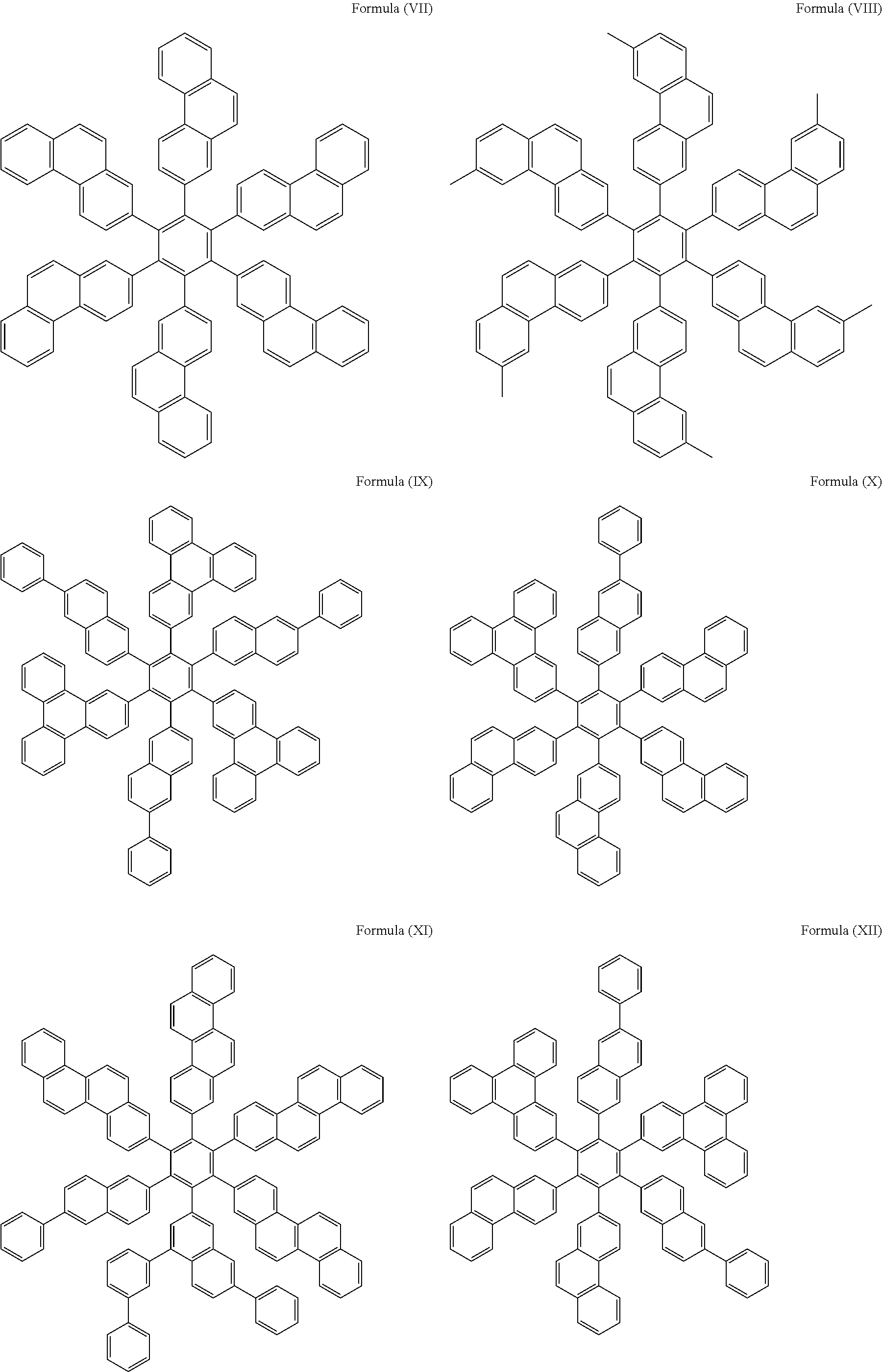

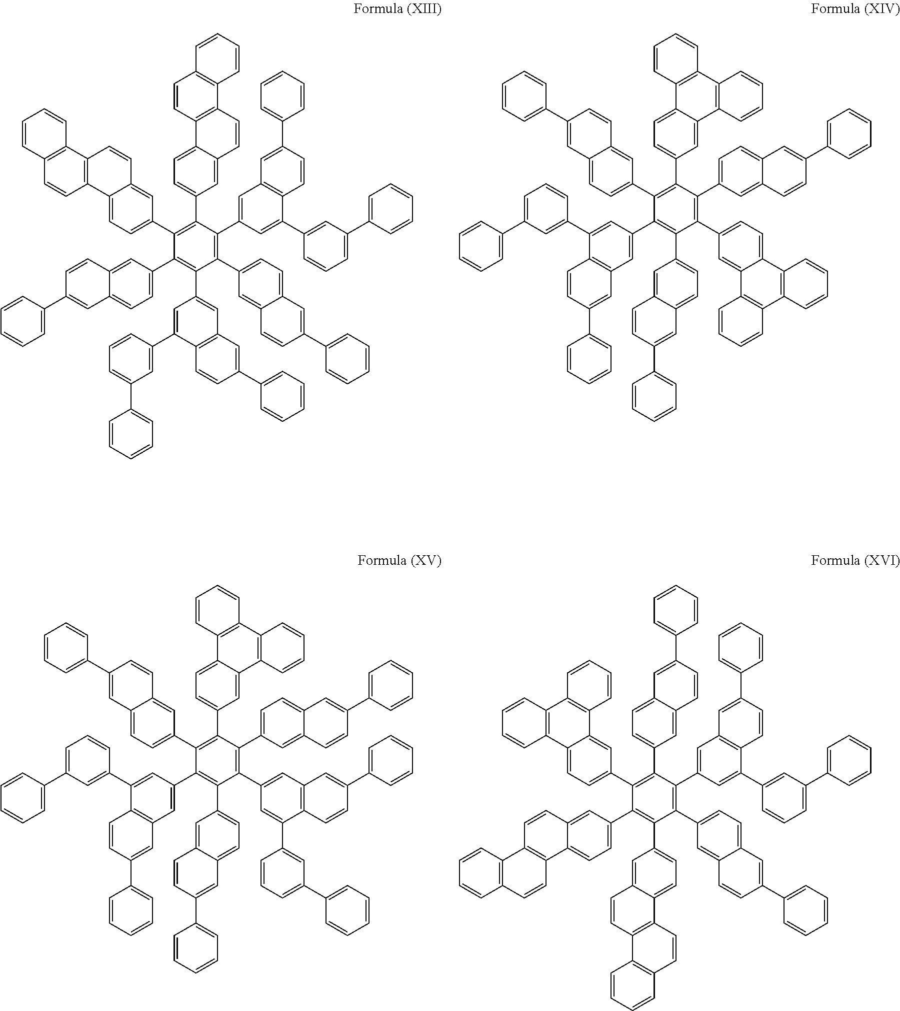

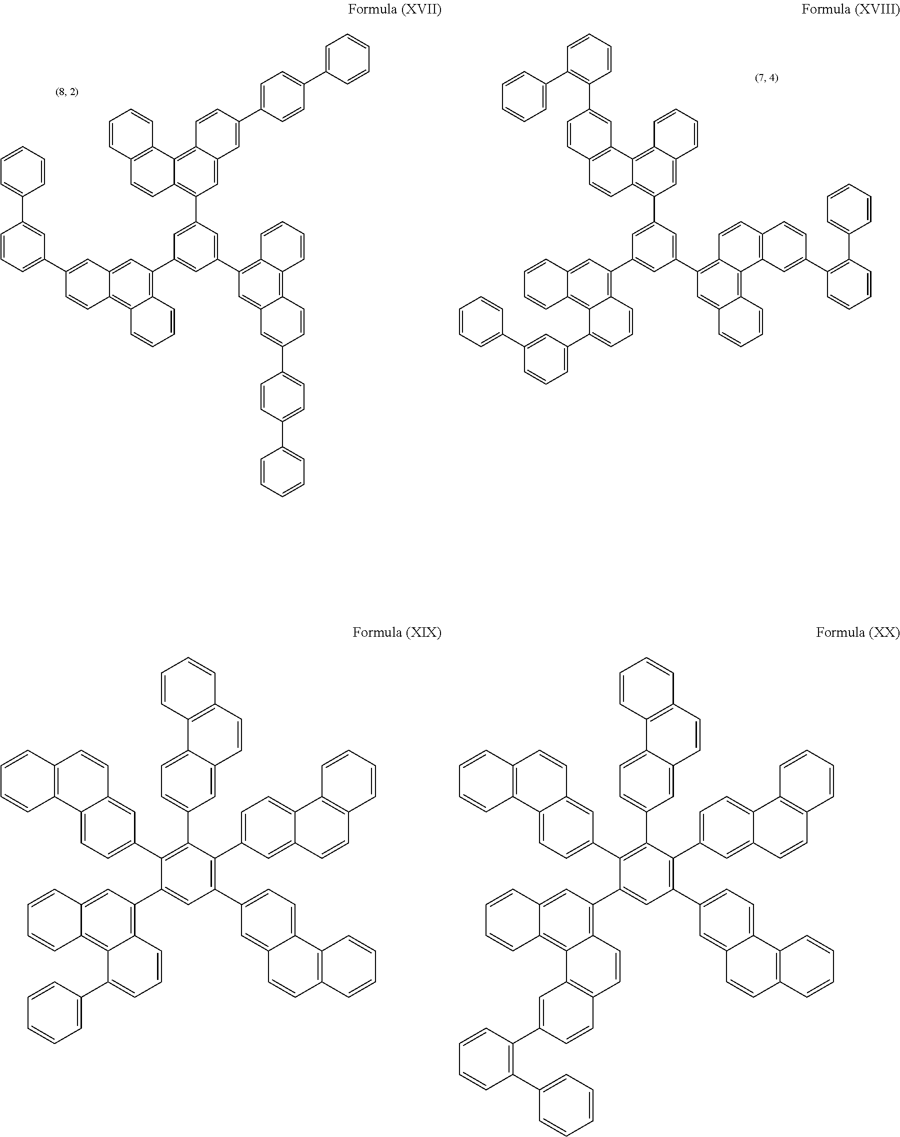

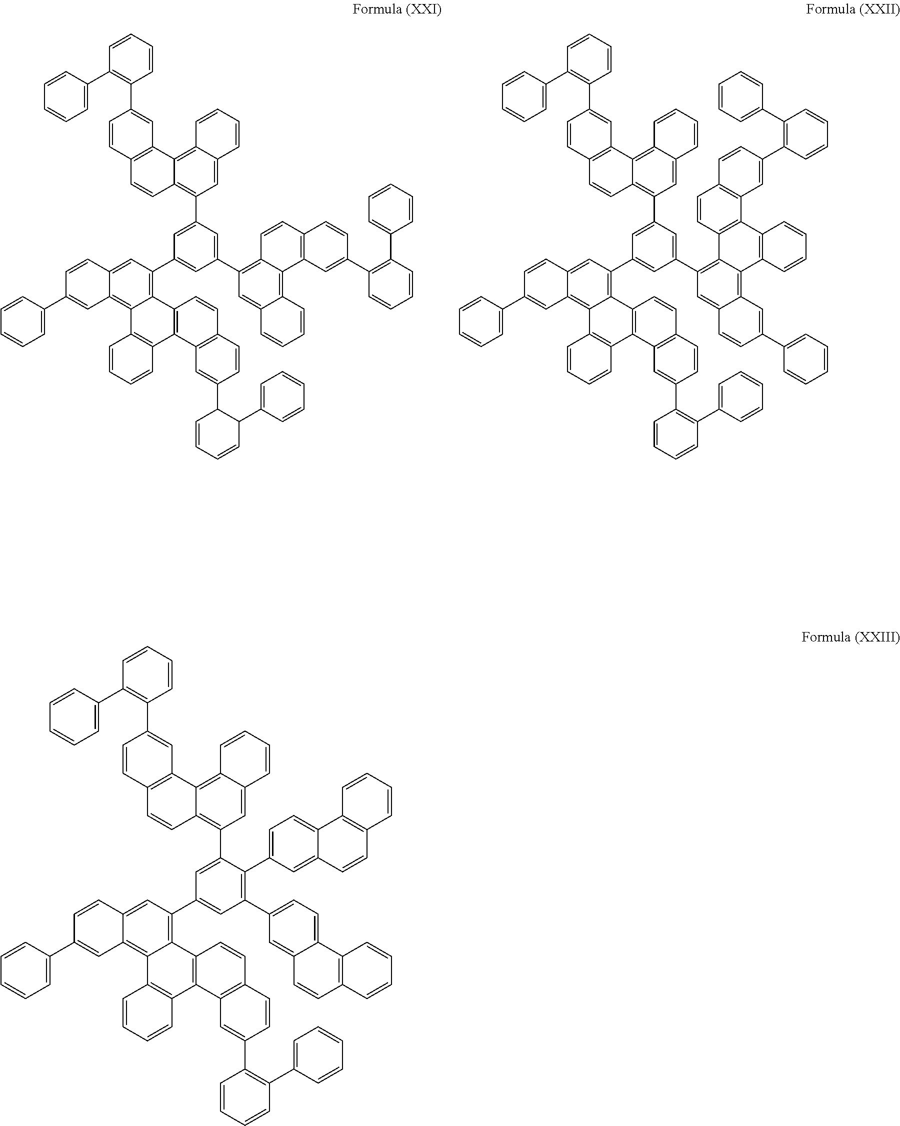

According to a further aspect, the present invention relates to the use of the polycyclic aromatic compounds of Formulas (I) to (XXIII) for preparing single wall carbon nanotubes.

The present invention will now be illustrated in further detail by the following Examples.

### EXAMPLES

### Characterization Methods

STM measurements were performed using low temperature Scanning Tunnelling Microscope in constant current mode and sample temperature of 77 K. The system is held in a separate UHV chamber with a base pressure of 1×10⁻¹¹ mbar. Raman spectra were recorded in a Bruker Senterra instrument with a spectral resolution of 4 cm⁻¹ using a 532 and 782 nm laser with a power of 20 mW. He Ions Scanning Microscopy measurements were performed in a Carl Zeiss Orion Plus instrument with a beam energy of 30 keV and a beam current 0.4 pA.

Preparation of Polycyclic Aromatic Compounds

Polycyclic aromatic compounds of Formulas (I) and (II) were prepared.

The compound of formula (I) was obtained by multistep organic synthesis according to synthetic route shown in FIG. 2. 2-Bromo-6-methylbenzophenanthrene (1) was obtained by standard Wittig Olefination of 2-acetonaphtone to the respective stilbene and subsequent Mallory-photocyclization using the Katz-improvement. Suzuki coupling of 1 with 2-biphenylboronic acid gave 2. Benzylic bromination with N-bromosuccinimide and following treatment with sodium cyanide in DMSO gave compound 4. The cyano-compound was then hydrolyzed to the corresponding acetic acid and converted to 6 in two steps. First reaction with thionylchloride gave the acid chloride which was then used in a Friedel-Crafts acylation to achieve ring closure. The last synthetic step, the conversion to compound of Formula (I) (referred to as 7 in FIG. 2), was carried out by aldol cyclotrimerization using either TiCl₄ or Brønsted acid conditions. Both cases lead to the desired compound in satisfying yield.

The compound of formula (II) was obtained by multistep organic synthesis according to synthetic route shown in FIG. 3. 2-methyl-triphenylene was prepared by Suzuki coupling of biphenyl-2-boronic acid and 4-bromotoluene resulting in 8 followed by Mallory-photocyclization using the Katz-improvement. Benzylic bromination with N-bromosuccinimide of 9 gave 2-bromomethyl-triphenylene which was subsequently converted to the corresponding Wittig salt 11 or phosphonate 12. Although stilbene 13 could not be obtained by Wittig reaction of 11 and acetonaphtone, the Homer-Emmons-Wadsworth reaction using 12 and 13 was successful. The following Mallory-photocyclization resulted in formation of the desired compound 14. The corresponding acetic acid 17 was then obtained by benzylic bromination of 14 with N-bromosuccinimide, a following treatment with potassium cyanide in the presence of tetrabutylammoniumbromide in CH₂Cl₂ and finally hydrolysis. 17 was then converted to the acid chloride by treatment with thionylchloride and subsequently Friedel-Crafts acylation was used to achieve ring closure and ketone 18 was obtained. For the trimerization of 18, reaction with TiCl₄ in o-DCB at 180° C. was carried out. The compound of Formula (II) (referred to as 19 in FIG. 3) was obtained.

Preparation of a Precursor Element for (6,6) Single Wall Carbon Nanotubes by Intramolecular Cyclization of Polycyclic Aromatic Compounds

The polycyclic aromatic compound of Formula (I) and Formula (II), respectively, was evaporated in UHV from a Knudsen-cell type evaporator at a rate of 0.5 Å/min on a previously cleaned Pt(111) surface at RT.

The Pt single crystal acquired from Surface Preparation Lab (SPL) was used as a cyclodehydrogenation catalyst. Surface was cleaned by standard sputtering with Ar ions with an energy of 1 KeV, first at room temperature and then at 1100K, followed by a last flash annealing at 1370 K without ion bombardment.

A post-annealing at about 200° C. was carried out. Further annealing at about 500° C. induces the complete surface-catalyzed cyclodehydrogenation of the molecules, thereby forming the desired precursor element for the (6,6) SWCNT to be prepared.

The precursor element is made of a SWCNT segment and a SWCNT cap which is attached to one end of the SWCNT segment, whereas the other end of the SWCNT segment remains open. The SWCNT segment is made of a segment ring (or rather two or more rings) formed by ortho-fused benzene rings. The structure of the precusor element is shown in FIG. 1. The SWCNT segment is represented by the grey shaded part, whereas the cap closing one end of the SWCNT segment is represented by the part without shading.

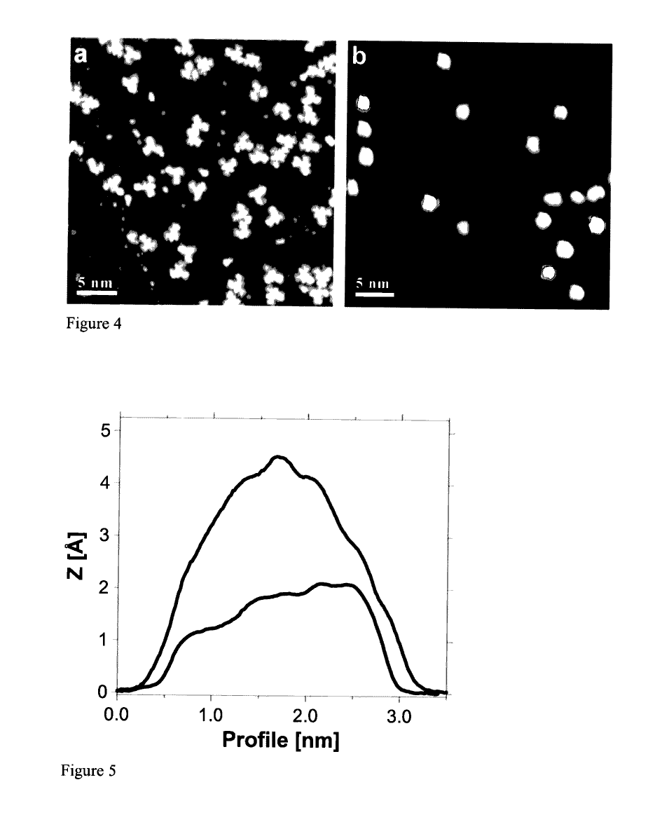

FIG. 4 shows a STM image of the polycyclic aromatic molecules deposited on the Pt surface at RT FIG. 4(a) and after Annealing at 500° C. FIG. 4(b). As the Pt single crystal and its surface are by far larger than the dimensions of the deposited molecules, the Pt surface reflects the situation of a more or less continuous and flat film on which the polycyclic aromatic molecules are applied.

FIG. 5 shows the line profiles of a polycyclic aromatic molecule prior to cyclodehydrogenation (solid line) and the precursor element obtained after cyclodehydrogenation (dotted line). As can be seen from FIG. 5, the height increases from about 0.2 nm (height of the more or less flat aromatic compound) to about 0.45 nm which is the height of the “bowl-like” precursor element.

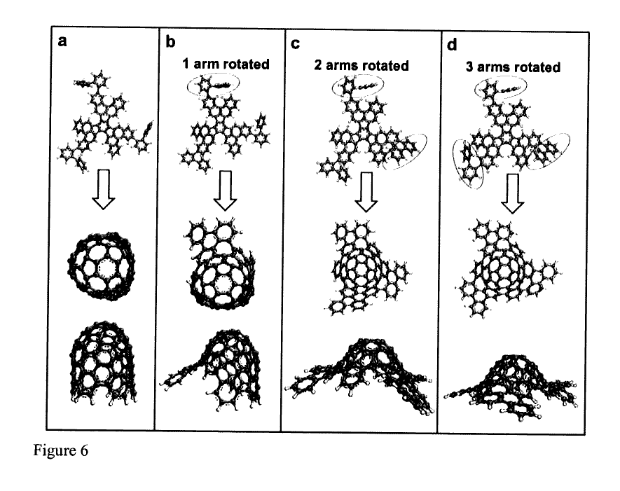

In FIG. 6, it is shown that different conformational isomers exist for the polycyclic aromatic compound of Formula (II). However, only one of these conformational isomers forms a curved bowl-like element made a SWCNT segment (in this particular case, a (6,6)-SWCNT segment) and a SWCNT cap. It is only this particular bowl-like element having the “correct” conformation which will subsequently grow in the CVD step. In FIG. 6, the conformational isomer on the very left forms a precursor element (shown from two different perspectives) which consists of a (6,6)-SWCNT segment and a cap attached to one end of the tube segment.

Growth of the Precursor Elements to Isomerically Pure (6,6) SWCNTs by Vapour Phase Reaction with Carbon-Source Molecules

The carbon source compound used for growing the precursor elements to the desired (6,6) SWCNTs was ethylene (C₂H₄) and ethanol (C₂H₅OH), respectively. A pressure of 1×10⁻⁷ mbar was maintained in the chamber. The substrates were annealed at 400° C. or 500° C. during 1 h. To have control on the low doses experiments, a pressure of 1×10⁻⁸ mbar was used.

For the CVD growth step, the precursor elements were left on the surface of the cyclodehydrogenation catalyst already used in step (i). So, the metal-containing catalyst of step (ii) was the same as used in step (i). As already mentioned above, the Pt single crystal catalyst and its surface are by far larger than the dimensions of the deposited molecules and the precursor elements prepared therefrom, which is why the Pt surface reflects the situation of a more or less continuous and flat catalyst film on which the isomerically pure SWCNTs are manufactured.

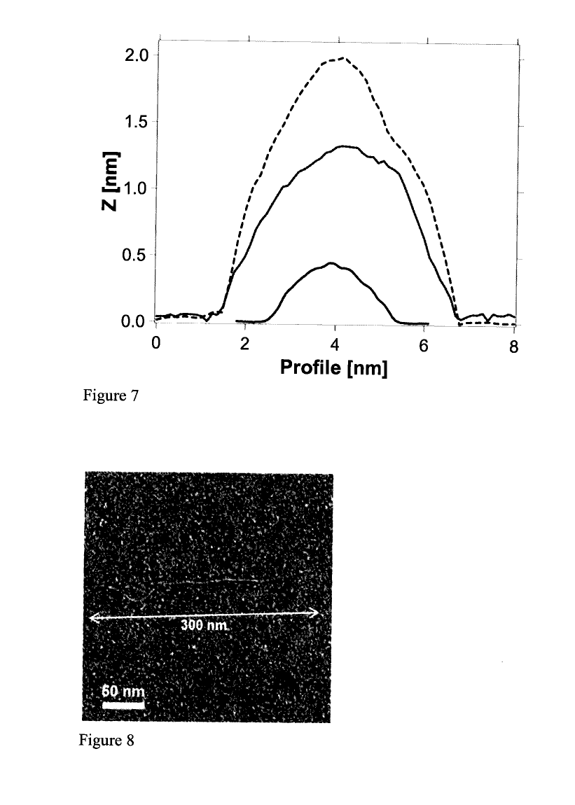

FIG. 7 shows the line profiles of the SWCNT precursor element before being reacted with the carbon source compound (solid line), and after having initiated the growth phase by feeding the carbon source compound (dotted line: dose of 1 Langmuir, dashed line: dose of 5 Langmuirs). As demonstrated by FIG. 7, a precursor element which consists of a (6,6)-SWCNT segment and a cap attached to one end of the tube segment will grow to the desired SWCNT by vapour phase reaction

FIG. 8 shows the scanning He ions microscopy image of a SWCNT of 300 nm in length.

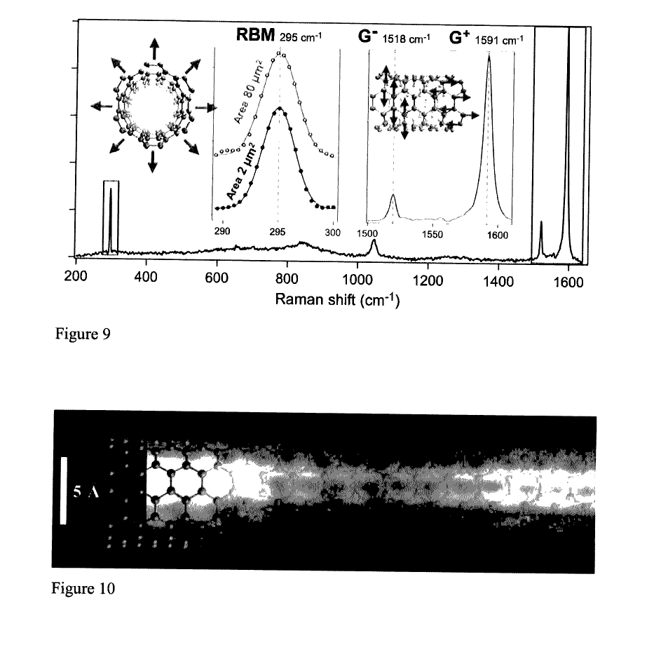

FIG. 9 shows the SWCNT Raman spectrum and is in conformity with monodisperse isomerically pure (6,6)-SWCNTs. The Raman spectrum presents very well defined bands at the expected positions for a (6,6) SWCNT. The extremely narrow band at 295 cm⁻¹ is associated to the Radial Breathing Mode (RBM) of a (6,6) tube. The RBM is a tangential out-of-plane acoustic mode which frequency depends strongly on the nanotube diameter. It is extremely important to outline that the spectrum does not show any further band within the RBM range (from 200-400 cm⁻¹), not even under illumination with a red laser with λ=782 nm, therefore supporting the extremely high selectivity of the process. In addition, the Raman spectrum presents the same bands when illuminating much bigger areas by using a lower magnifying objective. The inset of the figure shows the RBM band recorded by illuminating an area of 80 μm², thus assuring the measurement of a high number of SWCNT and therefore demonstrating the high selectivity of the process. The G band associated to the in-plane optical vibration of the graphene lattice appears as a double peak at 1518 and 1591 cm⁻¹. The significant curvature in small diameter SWCNT causes a shift to lower frequencies in the G peaks, especially for the vibrations associated to transversal (perpendicular to the tube axis) atomic displacements (G⁻), which appears downshifted with respect to the sp2 graphene lattice vibrations. The splitting observed is consistent with SWCNT of similar diameter and chirality. The additional peaks in the range from 400-1200 cm⁻¹ have been previously observed and used as an evidence for the presence of armchair SWCNT, since no peak in this range is present in semiconducting tubes.

The extremely cleanliness of the process reported here yields predefined chirality and defect-free SWCNT. A proof of that is the absence of any D band in the Raman spectrum.

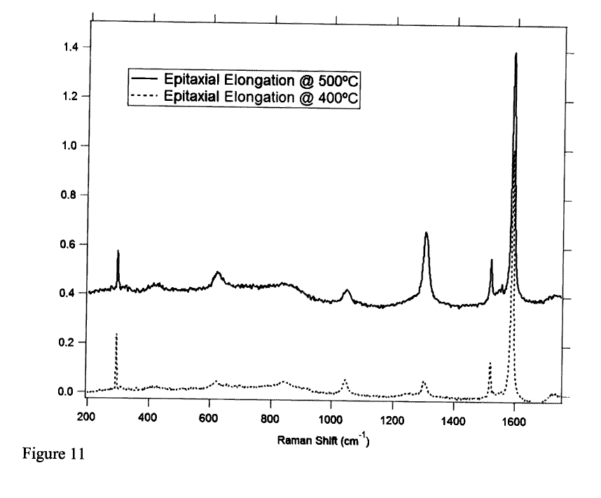

A high resolution STM picture of the SWCNT structure is shown in FIG. 10 which is a further proof for the true single chirality of the SWCNTs prepared in the present invention. In FIG. 10, it can be observed that the SWCNTs present an internal structure consisting in higher contrast lines in the direction of the tube axis. The model of the SWCNT is superimposed over the STM picture to further corroborate that those higher contrast lines are indeed the carbon positions of the graphene structure in a (6,6) SWCNT. The outstanding agreement in both diameter of the tube and periodicity of the graphene lattice demonstrate that the 1-D structures observed are the expected (6,6) SWCNTs.

Effect of Temperature in Step (ii) on Product Quality and Yield

FIG. 11 shows the Raman spectra of (6,6)-SWCNTs prepared at a temperature of 500° C. (solid line) and 400° C. (dotted line) in step (ii).

Both spectra are consistent with isomerically pure (6,6)-SWCNTs. However, by comparing the relative intensities between D/G bands and RBM/G, it can be concluded that the desired pre-defined SWCNTs can be obtained at higher yield when operating at lower temperature.

1.-17. (canceled)

18. A process for preparing single wall carbon nanotubes (SWCNT) having a diameter d_{SWCNT}, which comprises
(i) providing a precursor element which comprises a segment S_{SWCNT} of the single wall carbon nanotube,
the segment S_{SWCNT} being made of at least one ring formed by ortho-fused benzene rings, and having a first end E1 which is open and a second end E2 which is opposite to the first end E1,
the precursor element optionally further comprising a cap which is attached to the second end E2 of the segment S_{SWCNT},
(ii) growing the precursor element by vapour phase reaction with a carbon-source compound on the surface of a metal-containing catalyst, wherein the precursor element is in contact with the surface of the metal-containing catalyst via the open end E1 of the segment S_{SWCNT}, and the metal-containing catalyst is in the form of particles having an average diameter d_{cat} satisfying the following relation: d_{cat}>2×d_{SWCNT} or in the form of a continuous film.

19. The process according to claim 18, wherein the precursor element is prepared from a polycyclic aromatic compound.

20. The process according to claim 18, wherein the segment S_{SWCNT} is made of up to 10 rings, each ring being formed by ortho-fused benzene rings.

21. The process according to claim 18, wherein the precursor element is made of the segment S_{SWCNT} and the cap being attached to the second end E2 of the segment S_{SWCNT}.

22. The process according to claim 19, wherein the precursor element is prepared from the polycyclic aromatic compound by a surface-catalyzed intramolecular cyclisation.

23. The process according to claim 22, wherein the surface-catalyzed intramolecular cyclisation is carried out on the surface of a metal-containing catalyst.

24. The process according to claim 23, wherein the metal-containing catalyst of step (i) is in the form of particles having an average diameter d₆ satisfying the following relation: d_{cat}>2×d_{SWCNT} or in the form of a continuous film.

25. The process according to claim 23, wherein the particles of the metal-containing catalyst in step (i) have an average particle size of at least 5 nm.

26. The process according to claim 18, wherein the precursor element is prepared at a temperature T₁ of from 100° C. to 1000° C.

27. The process according to claim 18, wherein the carbon-source compound of step (ii) is selected from an alkane, an alkene, an alkyne, an alcohol, an aromatic compound, carbon monoxide, a nitrogen-containing organic compounds, a boron-containing organic compound, or any mixture thereof.

28. The process according claim 18, wherein the metal-containing catalyst of step (ii) comprises a metal selected from the group consisting of Pd, Pt, Ru, Ir, Rh, Au, Ag, Fe, Co, Cu, Ni, and mixtures or alloys thereof.

29. The process according to claim 18, wherein the particles of the metal-containing catalyst in step (ii) have an average particle size of at least 5 nm.

30. The process according to claim 23, wherein the precursor element is grown by vapour phase reaction with the carbon-source compound on the surface of the metal-containing catalyst of step (i).

31. The process according to claim 18, wherein step (ii) is carried out at a temperature T2 of 700° C. or less.

32. A single wall carbon nanotube, obtained by the process according to claim 18.

33. A polyaromatic compound having one of the following formulas (I) to (XXIII): 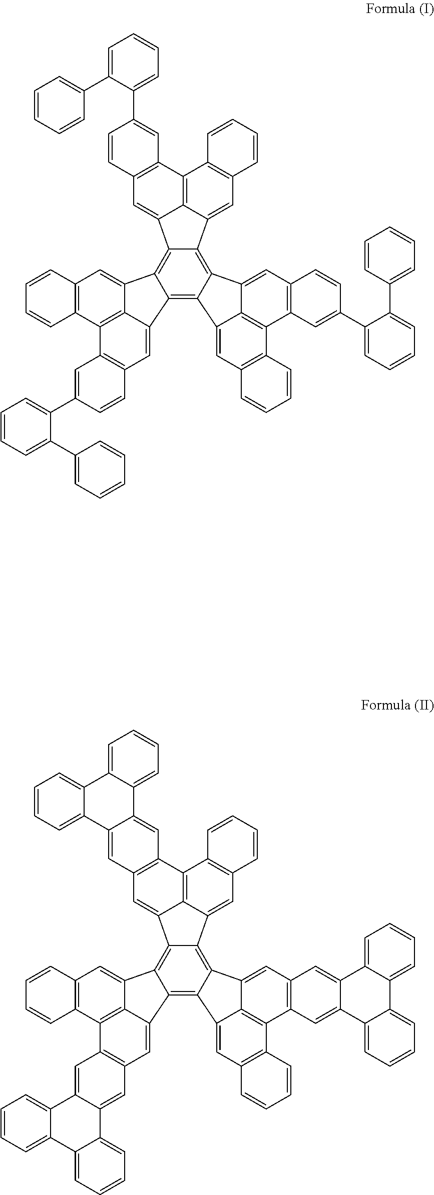 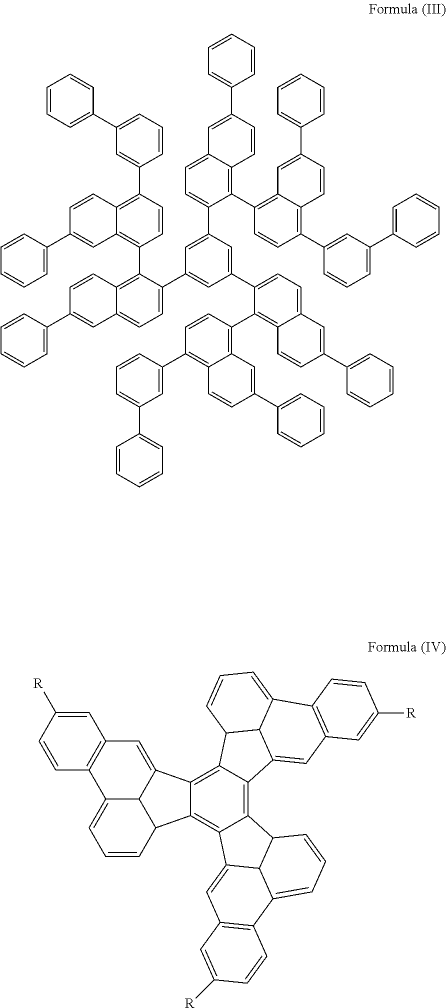
wherein R is phenyl (i.e. —C₆H₅);

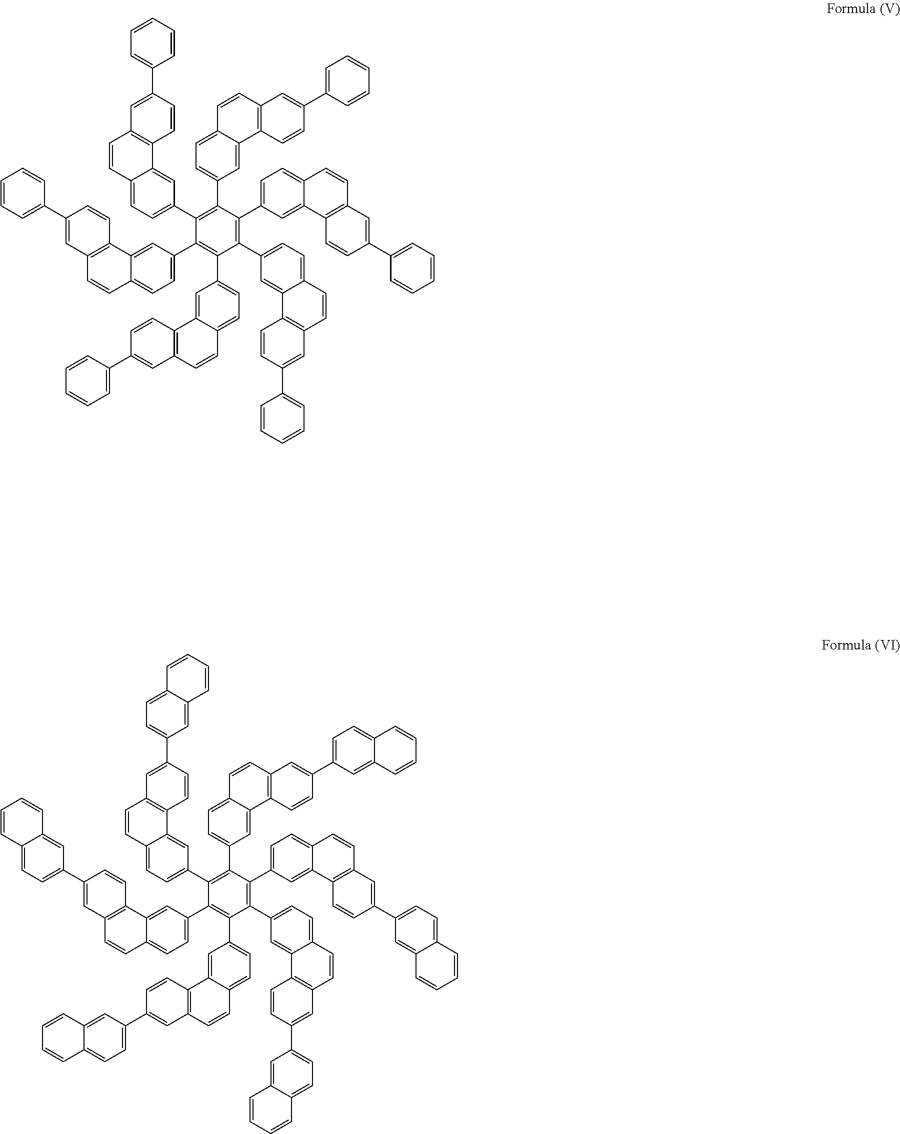

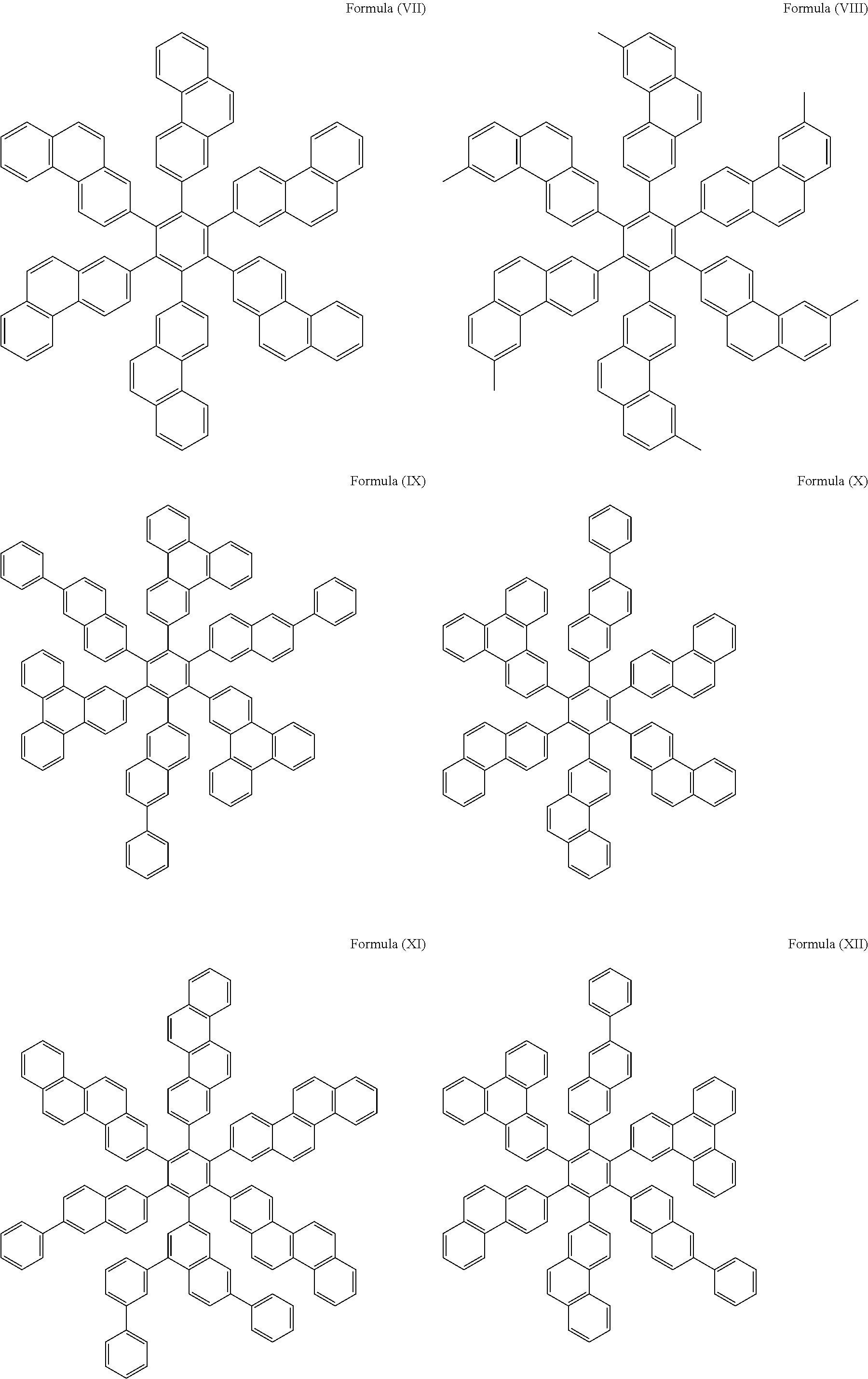

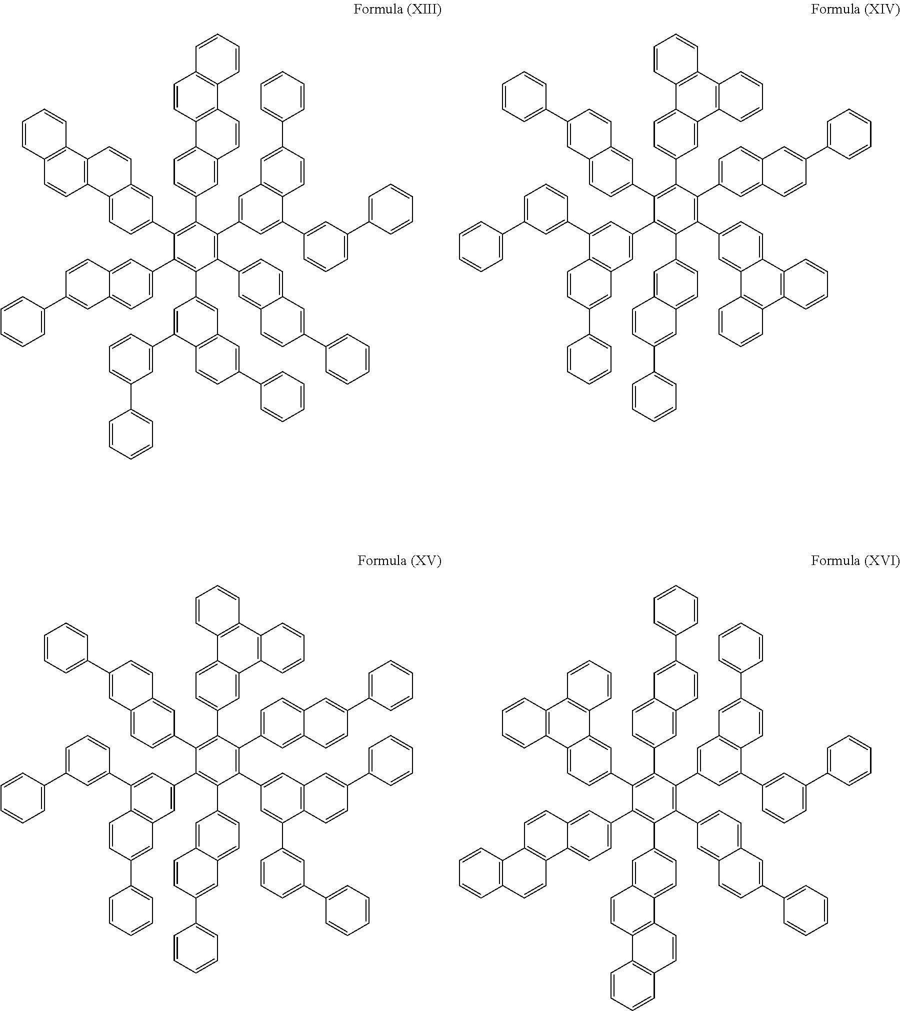

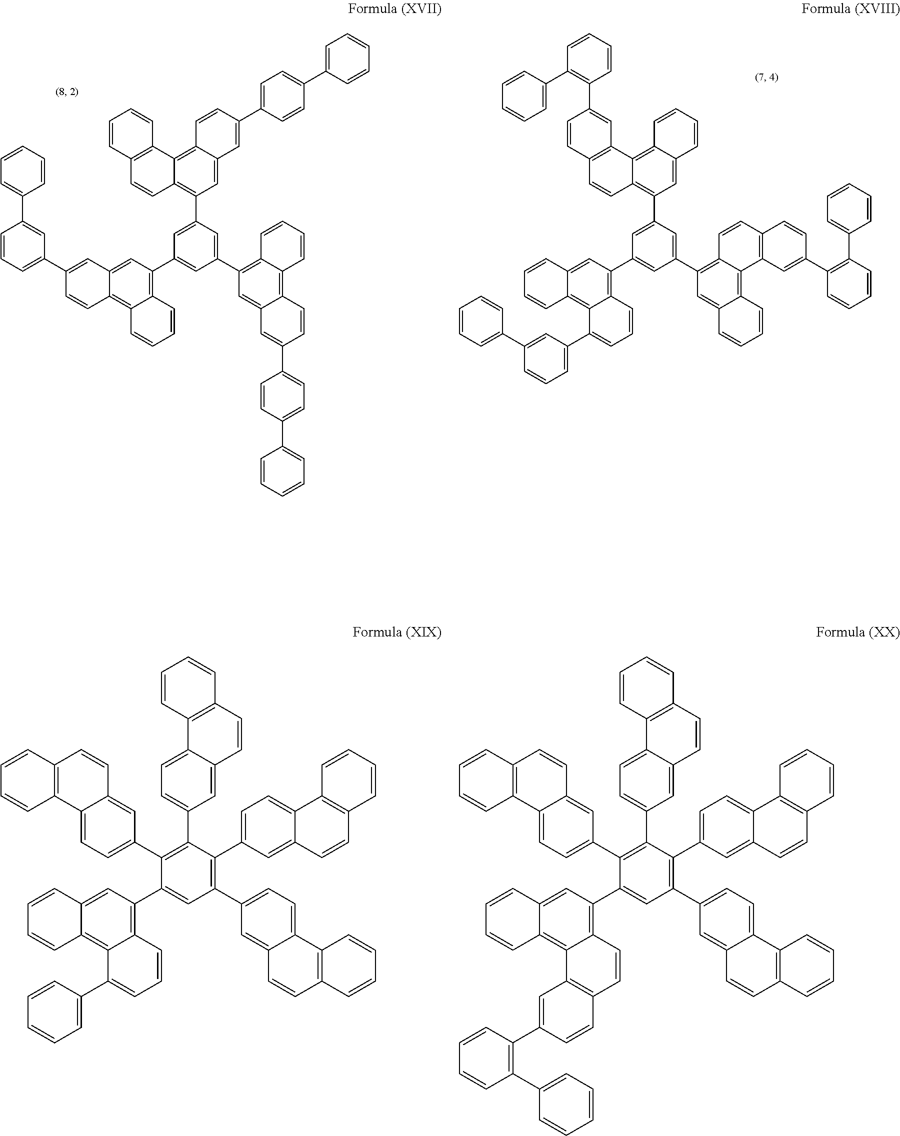

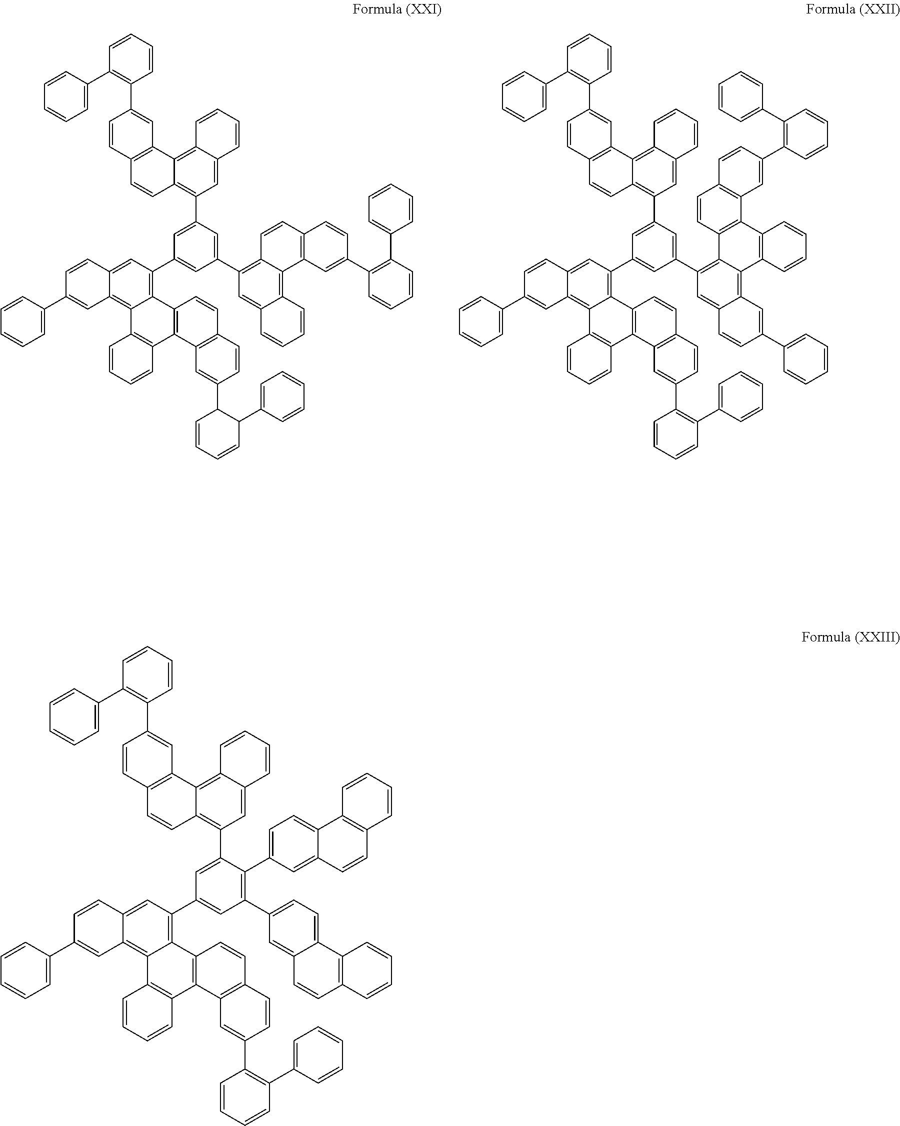

34. A method comprising preparing a single wall carbon nanotube from the polyaromatic compound of claim 33.

35. The process according to claim 23, wherein the metal is selected from the group consisting of Pd, Pt, Ru, Ir, Rh, Au, Ag, Fe, Co, Cu, Ni, and mixtures or alloys thereof.

36. The process according to claim 19, wherein the precursor element is prepared from the polycyclic aromatic compound by cyclodehydrogenation, cyclodehalogenation, or Bergman cyclization.
